(19) United States
(12) Patent Application Publication
(10) Pub. No.: US 2026/0364156 A1
(43) Pub. Date: Nov. 19, 2026

(54) DNA BARREL NANOSTRUCTURE VACCINES

(71) Applicants: President and Fellows of Harvard College, Cambridge, MA (US); Dana-Farber Cancer Institute, Inc., Boston, MA (US); Korea Institute of Science and Technology, Seoul (KR)

(72) Inventors: Yang C. Zeng, Winchester, MA (US); William M. Shih, Cambridge, MA (US); James l. MacDonald, Cambridge, MA (US); Ju Hee Ryu, Brookline, MA (US); lck Chan Kwon, Seoul (KR)

(73) Assignees: President and Fellows of Harvard College, Cambridge, MA (US); Dana-Farber Cancer Institute, Inc., Boston, MA (US); Korea Institute of Science and Technology, Seoul (KR)

(21) Appl. No.: 19/118,716

(22) PCT Filed: Oct. 6, 2023
(86) PCT No.: PCT/US2023/076197

§ 371 Date: Apr. 4, 2025

Related U.S. Application Data
(60) Provisional application No. 63/414,380, filed on Oct. 7, 2022.
(60) Provisional application No. 63/489,561, filed on Mar. 10, 2023.

(51) Int. Cl. A61K 39/00; A61K 39/39; A61K 45/06; A61P 35/00; B82Y 5/00

(52) U.S. Cl. CPC A61K 39/0011; A61K 39/39; A61K 45/06; A61P 35/00; B82Y 5/00; A61K 2039/53; A61K 2039/54; A61K 2039/545; A61K 2039/55555; A61K 2039/55561; A61K 2039/572; A61K 2039/6025; A61K 2039/62; A61K 2039/64

(57) ABSTRACT

The disclosure relates to nucleic acid-based vaccines for cancer and other diseases.

### RELATED APPLICATIONS

This application claims the benefit under 35 U.S.C. § 119 (e) of U.S. provisional application Ser. No. 63/414,380, filed Oct. 7, 2022, and U.S. provisional application Ser. No. 63/489,561, filed Mar. 10, 2023, each of which is incorporated by reference herein in its entirety.

### REFERENCE TO AN ELECTRONIC SEQUENCE LISTING

The contents of the electronic sequence listing (H049870783WO00-SEQ-MSB.xml; Size: 11,734 bytes; and Date of Creation on: Oct. 2, 2023) is herein incorporated by reference in its entirety.

### BACKGROUND

Neoantigens arise from nonsynonymous mutations in the cancer genome and are not subjected to central tolerance, making them potentially immunogenic. Vaccination against the tumor-specific neoantigens minimizes the potential induction of central and peripheral tolerance as well as the risk of autoimmunity. Neoantigen-based cancer vaccines have recently showed marked therapeutic potential in both preclinical and early-phase clinical studies. In situ vaccination, for example, takes advantage of the entire antigenic repertoire of a tumor to minimize immune escape, overcoming the limitations of conventional therapeutic cancer vaccines for melanoma and other cancers. Personalized cancer vaccination using nanomaterials to capture antigens ex vivo also holds great potential for cancer immunotherapy.

### SUMMARY

Therapeutic responses to neoantigen vaccines thus far have revealed limited cytotoxic CD8⁺ T cells but high frequencies of CD4⁺ T cells. An effective vaccine strategy, such as the one provided herein, targets neoantigens, which can minimize the risks of autoimmune reactions that may potentially arise from whole tumor cell or lysate vaccine approaches. Few studies provide feasible approaches for a vaccination strategy that captures neoantigens. Effective vaccination relies on, in some instances, administration of immune adjuvant to activate antigen presenting cells (APCs). For example, CpG oligodeoxynucleotides (CpG ODN) are regularly used adjuvants for the pattern recognition receptor TLR9 and are known to polarize anti-tumor immune responses. Studies indicate that nanoscale CpG spacing and density both play important roles for polarization to Th1 or Th2 immune response. CpG applied for vaccination along with local radiation in lymphoma patients also resulted in low level of CD8⁺ T cell proliferation. Understanding the role of adjuvant spacing and therapeutically exploiting the spatial distribution to control the immune response has been technically difficult, as current materials available to present these ligands typically only provide control over the average spacing and cannot offer precise and uniform spacing.

DNA origami (DNA organized into a three-dimensional structure, for example) can provide all the nanoparticle-related advantages for vaccination. Moreover, DNA origami uniquely enables investigations on the impact of spatial control over ligand as well as co-delivery of antigens and adjuvant to APCs. It was hypothesized that hydrophobicity of peptides was a major player in damage associated molecular pattern (DAMP) related signals. DNA origami can be fabricated with hydrophobic motif for neoantigen capturing, relying on the hydrophobicity of immunogenic neoantigens. Neoantigen-capturing motifs, for example, can be fabricated within a DNA origami structure shaped like a barrel (referred to herein simply as a “Barrel”) to avoid aggregation in the engineering process. Adjuvants can be fabricated on the surface of the Barrel, in some embodiments, to ensure optimal spacing for dendritic cell stimulation and Th1 polarized immune response. In the present disclosure, Barrels with optimal adjuvant (e.g., CpG and/or dsRNA) spacing were fabricated and neoantigen-capturing motifs in Barrels were fabricated. This nucleic acid origami vaccine platform (also referred to as DoriVac) enables co-presentation of captured neoantigens and optimally spaced adjuvants to APCs for a neoantigen-specific Th1 polarized immune response and improved cancer therapeutic efficacy. Specifically, in some embodiments, the DNA origami vaccine comprises nucleic acid Barrel nanostructure conjugated to adjuvant molecules and antigen-capturing motifs. In some embodiment, the DNA origami vaccine comprises nucleic acid Barrel nanostructure conjugated to antigen molecules and adjuvant molecules. Furthermore, in some embodiments, this DNA origami vaccine platform enables co-presentation of captured neoantigens and optimally spaced adjuvants to APCs systematically or in situ.

The vaccine platform provided herein is highly translational. The DNA origami itself has limited immunogenicity. The other components (e.g., adjuvants such as CpG and double-stranded RNA (dsRNA), and/or antigen-capturing motifs such as coiled coil peptides) that are used herein have previously been used in a clinical setting. This DNA origami vaccine platform may be used for many cancer types, and in combination with other chemo- or immuno-therapeutics. Furthermore, this DNA origami vaccine platform may be used for vaccination against other diseases (e.g., infectious diseases)

### BRIEF DESCRIPTION OF THE DRAWINGS

FIG. 1: Basic Barrel design. The DNA double helices were arranged into three layers and bent to fold closed barrel.

FIGS. 2A-2B: Design of DNA origami Barrel for payload fabrication. (A) Schematic picture showing the modification location of the double helices for different cargos. Double helices are numbered for reference in the CaDNAno software program. (B) Modification sites for handles as demonstrated by the CaDNAno DNA routing. Pink: Cy5, green: CpG, purple: dsRNA, blue: CCP.

FIGS. 3A-3D: Barrel fabrication optimization (A) Folding condition optimization for barrel (B) TEM images at low and high magnification showing the monodispersity and robustness of the barrel production. (C) Purification optimization with different concentration of PEG buffer. (D) Barrel folding with CpG and dsRNA at different excess.

FIGS. 4A-4D: DNA origami Barrel fabricated with dsRNA and CpG. (A) TEM and model images of Barrels. Size of Barrel: 30×60 nm. (B) Modification sites on the Barrel outer surface for Cy5, dsRNA and CpG. (C) Modification sites inside of the Barrel for the coiled-coil motif. (D) Conjugation of CpG and dsRNA (25 nt) to the Barrel with various excess concentrations. The conjugation was successful.

FIGS. 5A-5B: Barrel fabricated with CpG and dsRNA stimulated HEK blue cells. (A) HEK blue detection of TLR9 expression cells activated by various Barrel structures and controls. (B) HEK blue detection of TLR3 expression cells activated by various Barrel structures and controls.

FIGS. 6A-6G: Coiled-coil peptides (CCPs) can be fabricated successfully in the Barrel and demonstrate self-capturing. (A) Amino acid sequence, location of DNA attachment, and net charge of the peptides used in this study (azK indicates azidolysine). (B) Denaturing PAGE gel results showed that PBS and 5× excess of peptide serve as good condition for CCP-oligo conjugation. (C) Pure peptide-DNA conjugates were achieved by PAGE purification as illustrated by a single band. Increasing amounts of product were loaded per well from left to right. (D) All 5 peptides were successfully conjugated to the Barrel by handle/anti-handle DNA hybridization. I TEM images of the Barrels at different conditions. After conjugation of CCP-E4_N (right panel), a decrease in blank area inside the barrel was observed (white space) compared to the barrel (left) and the barrel with CpG and dsRNA (left and middle panel). Size of Barrel: 30×60 nm. (F) Agarose gel results showing the Barrels were fabricated with different excess of CCPs. B-C-R: Barrel-CpG-dsRNA. 1×P means equal molar ratio of CCP without anti-handle was incubated with Barrels. (G) After DNase digestion, the peptide conjugation efficiency was determined by sliver staining of SDS PAGE gel. Increased peptide intensity verified the self-capturing of more CCPs once the CCP is fabricated inside the barrel.

FIGS. 7A-7C: Barrel vaccine platform captures short peptides. (A) Silver stain of SDS page gel showing the CCP captured hydrophobic peptides after using DNase to digest all the barrel structures. Upper band: DNase I. Lower band: CCP+short peptides (hydrophobicity: FGFGF>RGFGY>GGFGG). (B) Quantification of the band intensity. (C) HPLC results showing the remaining peptides in the supernatant after precipitate the Barrel vaccine nanoparticle. The less the bar shows, the more peptides were captured by Barrel vaccine platform.

FIGS. 8A-8D: Barrel vaccine platform captures proteins released by irradiated tumor cell. (A) A schematic showing how cells are irradiated and how the Barrel vaccine platform captures proteins were analyzed. (B, C) Images showing CT26 and B16F10 cells before and after irradiation. (D) Silver stain results showing the captured proteins running through 4-12% SDS page gel.

FIGS. 9A-9F: Mass spectroscopic analysis of the captured proteins. (A-D) A preliminary testing using mass spectrometry to check the barrel captured antigens that may related to the improved vaccination. (E-F) Mass spectroscopic investigation comparing the amount of antigens being captured in with different Barrel conditions.

FIGS. 10A-10D: Barrel DNA origami vaccine (DoriVac) efficacy. (A) B16F10 tumor model setup and treatment scheme. (B) B16F10 tumors images on Day 14 after 3 doses of vaccination and 5 doses of anti-PD-L1 if applied. (C) Mouse tumor growth curve (n=7). (D) Mouse survival curve (n=7).

FIGS. 11A-11F: Immune cell profiling. (A, B) CD40 and PD-L1 expression levels on the CD11c positive dendritic cells in the draining lymph nodes analyzed by flow cytometry. (C, D) CD69 expression levels on the CD4 and CD8 positive T cells in the draining lymph nodes analyzed by flow cytometry. (E, F) IFNγ expression levels on the CD4 and CD8 positive T cells in the draining lymph nodes analyzed by flow cytometry.

FIGS. 12A-12E: Barrel DoriVac efficacy with less B16F10 cell inoculation. (A) B16F10 tumor model setup and treatment scheme. (B) B16F10 tumors images on Day 17 after 3 doses of vaccination and 5 doses of anti-PD-L1 if applied. (C) Mouse tumor growth curve (n=7). (D) Mouse survival curve (n=7). (E) Mouse survival curve after tumor rechallenge with 1×10⁵ B16F10 tumor cells to the survived mice from D. Controls are naïve mice receiving the same number of cells.

FIGS. 13A-13D: Barrel DoriVac efficacy as applied for in situ vaccination. (A) B16F10 tumor model setup and treatment scheme. Doxorubicin (4 mg/kg) was applied intramurally to induce immunogenic cell death locally. Barrel DoriVac (without antigen pre-captured) was applied intramurally and anti-PD-L1 was administered surrounding the tumor tissue. (B) B16F10 tumors images on Day 12 after 1 doses of vaccination and 3 doses of anti-PD-L1 if applied. (C) Mouse survival curve (n=7). (D) Mouse survival curve after tumor rechallenge with 1×10⁵ B16F10 tumor cells to surviving mice from (C). Control mice are naïve mice receiving the same number of cells.

FIGS. 14A-14D: Barrel DoriVac efficacy with MC38 colon carcinoma model. (A) MC38 tumor model setup and treatment scheme. (B) MC38 tumors images on Day 15 after 3 doses of vaccination and 5 doses of anti-PD-L1 if applied. (C) Mouse survival curve (n=7). (D) Mouse survival curve after tumor rechallenge with 2×10⁵ MC38 tumor cells to surviving mice from (C). Controls are naïve mice receiving the same number of cells.

FIGS. 15A-15B: Prophylactic vaccination. (A) C57BL6 mice received two doses of vaccines on Day 0 and Day 7. 1×10⁵ B16F10 cells were applied to inoculate the mice on Day 7. Mouse survival was recorded (n=5). (B) C57BL6 mice received two doses of vaccines on Day 0 and Day 7. 2×10⁵ MC38 cells were applied to inoculate the mice on Day 7. Mouse survival was recorded (n=5).

### DETAILED DESCRIPTION

Provided herein are nucleic acid nucleic acid nanostructure vaccines comprising nucleic acid nanostructures (e.g., nucleic acid barrel nanostructures) that are linked to (e.g., conjugated to) adjuvant molecules, antigen molecules and/or antigen-capturing motifs. In some embodiments, provided herein are nucleic acid barrel nanostructures conjugated to adjuvant molecules and antigen molecules. Further embodiments provide nucleic acid barrel nanostructures conjugated to adjuvant molecules and antigen-capturing motifs (e.g., coiled-coil peptides (CCPs)).

In some embodiments, a nucleic acid nanostructure comprises at least 5, at least 10, or at least 20 adjuvant (e.g., CpG and/or double-stranded RNA (dsRNA)) molecules. In some embodiments, a nucleic acid nanostructure comprises at least 5 (e.g., CpG and/or dsRNA) molecules. For example, a nucleic acid nanostructure may comprise 5-200, 5-175, 5-150, 5-125, 5-100, 5-85, 5-75, 5-65, 5-55, 5-50, 5-45, 5-40, 5-35, 5-30, 5-35, 5-20, 10-55, 10-50, 10-45, 10-40, 10-35, 10-30, 10-35, 10-20, 15-55, 15-50, 15-45, 15-40, 15-35, 15-30, 15-35, or 15-20 adjuvant (e.g., CpG and/or dsRNA) molecules. In some embodiments, the nucleic acid nanostructure comprises 100 to 200, 150 to 200, 100 to 150, 50 to 100, 25 to 75, 25 to 50, 5 to 25, 10 to 25, or 15 to 25 adjuvant (e.g., CpG and/or dsRNA) molecules. In some embodiments, a nucleic acid nanostructure comprises 5, 6, 7, 8, 9, 10, 11, 12, 13, 14, 15, 16, 17, 18, 19, 20, 21, 22, 23, or 24 adjuvant (e.g., CpG and/or dsRNA) molecules. In some embodiments, a nucleic acid nanostructure comprises 18 adjuvant (e.g., CpG and/or dsRNA) molecules.

In some embodiments, each adjuvant molecule of the plurality of adjuvant molecules is uniformly spaced 4.0 nm, 4.5 nm, 5 nm, 5.5 nm, 6 nm, 6.5 nm, or 7 nm from any other adjacent adjuvant molecule. In some embodiments, each adjuvant molecule of the plurality of adjuvant molecules is uniformly spaced 4.3 nm from any other adjacent adjuvant molecule 4 nm-10 nm, 4.5 nm-10 nm, 5 nm-10 nm, 5.5 nm-10 nm, 6 nm-10 nm, 6.5 nm-10 nm, 7 nm-10 nm, 7.5 nm-10 nm, 8 nm-10 nm, 8.5 nm-10 nm, 9 nm-10 nm, 9.5 nm-10 nm from any other adjacent adjuvant molecule.

In some aspects, the present disclosure provides a nucleic acid nanostructure comprising a plurality of adjuvant molecules and a plurality of antigens, wherein each adjuvant molecule of the plurality of adjuvant molecules is uniformly spaced about 4.3 nm from any other adjuvant molecule.

In some embodiments, a nucleic acid nanostructure comprises at least 5, at least 10, at least 20, or at least 40 antigen-capturing motifs (e.g., coiled-coil peptides (CCPs). In some embodiments, a nucleic acid nanostructure comprises at least 5 antigen-capturing motifs. For example, a nucleic acid nanostructure may comprise 5-55, 5-50, 5-45, 5-40, 5-35, 5-30, 5-35, 5-20, 10-55, 10-50, 10-45, 10-40, 10-35, 10-30, 10-35, 10-20, 15-55, 15-50, 15-45, 15-40, 15-35, 15-30, 15-35, or 15-20 antigen-capturing motifs. In some embodiments, the nucleic acid nanostructure comprises 100 to 200, 150 to 200, 100 to 150, 50 to 100, 25 to 75, 25 to 50, 5 to 25, 10 to 25, or 15 to 25 antigen-capturing motifs. In some embodiments, a nucleic acid nanostructure comprises 5, 6, 7, 8, 9, 10, 11, 12, 13, 14, 15, 16, 17, 18, 19, 20, 25, 30, 40, 42, or 45 antigen-capturing motifs.

In some embodiments, each antigen-capturing motif (e.g., CCP) of the plurality of antigen-capturing motif is uniformly spaced 4.0 nm, 4.5 nm, 5 nm, 5.5 nm, 6 nm, 6.5 nm, or 7 nm from any other adjacent antigen-capturing motif. In some embodiments, each antigen-capturing motif of the plurality of antigen-capturing motifs is spaced at 8.6-10 nm from any other adjacent antigen-capturing motif 4 nm-50 nm, 5 nm-50 nm, 4 nm-40 nm, 4 nm-30 nm, 4 nm-25 nm, 4 nm-20 nm, 4 nm-15 nm, 5 nm-15 nm, 4 nm-10 nm, 4.5 nm-10 nm, 5 nm-10 nm, 5.5 nm-10 nm, 6 nm-10 nm, 6.5 nm-10 nm, 7 nm-10 nm, 7.5 nm-10 nm, 8 nm-10 nm, 8.5 nm-10 nm, 9 nm-10 nm, 9.5 nm-10 nm from any other adjacent antigen-capturing motif.

In some aspects, the present disclosure provides a nucleic acid nanostructure comprising a plurality of antigen-capturing motifs (e.g., CCPs) and a plurality of antigens, wherein each antigen-capturing motif of the plurality of antigen-capturing motifs is spaced at 8.6-10 nm away from any other antigen-capturing motif.

In some aspects, the present disclosure provides a nucleic acid nanostructure comprising a plurality of adjuvant molecules, a plurality of antigen-capturing motifs, and/or a plurality of antigens, wherein each adjuvant molecule of the plurality of adjuvant molecules and each antigen-capturing motif of the plurality of antigen-capturing motifs is uniformly spaced about 8.6-10 nm from any other adjuvant molecule and/or antigen-capturing motif.

In some embodiments, the distance between any two adjacent molecules of adjuvant (e.g., CpG and/or dsRNA) is 2 nm-10 nm. For example, the distance between any two adjacent molecules of adjuvant (e.g., CpG and/or dsRNA) may be about 4-10 nm, 4-8 nm, or about 4-6 nm. In some embodiments, the distance between any two adjacent molecules of adjuvant is 4 nm-50 nm, 5 nm-50 nm, 4 nm-40 nm, 4 nm-30 nm, 4 nm-25 nm, 4 nm-20 nm, 4 nm-15 nm, 5 nm-15 nm, 4 nm-10 nm, 4.5 nm-10 nm, 5 nm-10 nm, 5.5 nm-10 nm, 6 nm-10 nm, 6.5 nm-10 nm, 7 nm-10 nm, 7.5 nm-10 nm, 8 nm-10 nm, 8.5 nm-10 nm, 9 nm-10 nm, 9.5 nm-10 nm. In some embodiments, the distance between any two adjacent molecules of adjuvant (e.g., CpG and/or dsRNA) is a 4.0, 4.1, 4.2, 4.3, 4.4, 4.5, 4.6, 4.7, 4.8, 4.9, or 5.0 nm. Uniform spacing herein refers to the distance between any two adjacent molecules, measuring from the center of the molecule.

In some embodiments, the density of adjuvant (e.g., CpG and/or dsRNA) on the nucleic acid nanostructure is 1 molecule of adjuvant (e.g., CpG and/or dsRNA) per 5 to 50 nm². In some embodiments, the density of adjuvant (e.g., CpG and/or dsRNA) on the nucleic acid nanostructure is 1 molecule of adjuvant per 5 to 10 nm², 1 molecule of adjuvant per 5 to 20 nm², 1 molecule of adjuvant per 5 to 30 nm², 1 molecule of adjuvant per 5 to 40 nm², 1 molecule of adjuvant per 10 to 50 nm², 1 molecule of adjuvant per 20 to 50 nm², 1 molecule of adjuvant per 30 to 50 nm², 1 molecule of adjuvant per 40 to 50 nm², or 1 molecule of adjuvant per 20 to 40 nm². In some embodiments, the density of adjuvant (e.g., CpG and/or dsRNA) on the nucleic acid nanostructure is 1 molecule per 5 nm², 1 molecule per 10 nm², 1 molecule per 15 nm², 1 molecule per 20 nm², 1 molecule per 25 nm², 1 molecule per 30 nm², 1 molecule per 35 nm², 1 molecule per 40 nm², 1 molecule per 45 nm², or 1 molecule per 50 nm².

In some embodiments, the distance between any two adjacent molecules of adjuvant (e.g., adjuvant molecule) (e.g., CpG and/or dsRNA) is about 4.3 nm. In some embodiments, the density of adjuvant (e.g., adjuvant molecule) (e.g., CpG and/or dsRNA) on the nucleic acid nanostructure is 1 molecule of adjuvant (e.g., CpG and/or dsRNA) per 5 to 10 nm². In some embodiments, the density of adjuvant (e.g., CpG and/or dsRNA) on the nucleic acid nanostructure is 1 molecule of adjuvant (e.g., CpG and/or dsRNA) per 5, 5.5, 6, 6.5, 7, 7.5, 8, 8.5, 9, 9.5, or 10 nm².

In some embodiments, the distance between any two adjacent antigen-capturing motifs is 2-10 nm. For example, the distance between any two adjacent antigen-capturing motifs may be about 4-10 nm, 4-8 nm, or about 4-6 nm. In some embodiments, the distance between any two adjacent antigen-capturing motifs is 4 nm-50 nm, 5 nm-50 nm, 4 nm-40 nm, 4 nm-30 nm, 4 nm-25 nm, 4 nm-20 nm, 4 nm-15 nm, 5 nm-15 nm, 4 nm-10 nm, 4.5 nm-10 nm, 5 nm-10 nm, 5.5 nm-10 nm, 6 nm-10 nm, 6.5 nm-10 nm, 7 nm-10 nm, 7.5 nm-10 nm, 8 nm-10 nm, 8.5 nm-10 nm, 9 nm-10 nm, 9.5 nm-10 nm. In some embodiments, the distance between any two adjacent antigen-capturing motifs is a 4.0, 4.1, 4.2, 4.3, 4.4, 4.5, 4.6, 4.7, 4.8, 4.9, or 5.0 nm. Uniform spacing herein refers to the distance between any two adjacent molecules, measuring from the center of the molecule or from the point of conjugation to the nanostructure.

In some embodiments, the density of antigen-capturing motifs on the nucleic acid nanostructure is 1 antigen-capturing motif per 5 to 50 nm². In some embodiments, the density of antigen-capturing motifs on the nucleic acid nanostructure is 1 antigen-capturing motif per 5 to 10 nm², 1 antigen-capturing motif per 5 to 20 nm², 1 antigen-capturing motif per 5 to 30 nm², 1 antigen-capturing motif per 5 to 40 nm², 1 antigen-capturing motif per 10 to 50 nm², 1 antigen-capturing motif per 20 to 50 nm², 1 antigen-capturing motif per 30 to 50 nm², 1 antigen-capturing motif per 40 to 50 nm², or 1 antigen-capturing motif per 20 to 40 nm². In some embodiments, the density of antigen-capturing motifs on the nucleic acid nanostructure is 1 antigen-capturing motif per 5 nm², 1 antigen-capturing motif per 10 nm², 1 antigen-capturing motif per 15 nm², 1 antigen-capturing motif per 20 nm², 1 antigen-capturing motif per 25 nm², 1 antigen-capturing motif per 30 nm², 1 antigen-capturing motif per 35 nm², 1 antigen-capturing motif per 40 nm², 1 antigen-capturing motif per 45 nm², or 1 antigen-capturing motif per 50 nm².

In some embodiments, the distance between any two adjacent molecules of adjuvant (e.g., CpG and/or dsRNA) and/or CCPs is 2 nm-10 nm. For example, the distance between any two adjacent molecules of adjuvant (e.g., CpG and/or dsRNA) and/or CCPs may be about 4-10 nm, 4-8 nm, or about 4-6 nm. In some embodiments, the distance between any two adjacent molecules of adjuvant (e.g., CpG and/or dsRNA) and/or CCPs is a 4.0, 4.1, 4.2, 4.3, 4.4, 4.5, 4.6, 4.7, 4.8, 4.9, or 5.0 nm. Uniform spacing herein refers to the distance between any two adjacent molecules, measuring from the center of the molecule.

In some embodiments, the density of adjuvant (e.g., CpG and/or dsRNA) and/or CCPs on the nucleic acid nanostructure is 1 molecule of adjuvant (e.g., CpG and/or dsRNA) or CCP per 5 to 50 nm². In some embodiments, the density of adjuvant (e.g., CpG and/or dsRNA) and/or CCP on the nucleic acid nanostructure is 1 molecule per 5 nm², 1 molecule per 10 nm², 1 molecule per 15 nm², 1 molecule per 20 nm², 1 molecule per 25 nm², 1 molecule per 30 nm², 1 molecule per 35 nm², 1 molecule per 40 nm², 1 molecule per 45 nm², or 1 molecule per 50 nm².

In some embodiments, the distance between any two adjacent molecules of adjuvant (e.g., adjuvant molecule) (e.g., CpG and/or dsRNA) and/or CCPs is about 4.3 nm. In some embodiments, the density of adjuvant (e.g., adjuvant molecule) (e.g., CpG and/or dsRNA) and/or CCP on the nucleic acid nanostructure is 1 molecule of adjuvant (e.g., CpG and/or dsRNA) or CCP per 5 to 10 nm². In some embodiments, the density of adjuvant (e.g., CpG and/or dsRNA) and/or CCP on the nucleic acid nanostructure is 1 molecule of adjuvant (e.g., CpG and/or dsRNA) or CCP per 5, 5.5, 6, 6.5, 7, 7.5, 8, 8.5, 9, 9.5, or 10 nm².

In some embodiments, the nucleic acid nanostructure is a nucleic acid (e.g., DNA) origami nanostructure. In some embodiments, the nucleic acid nanostructure is a nucleic acid (e.g., DNA) single-stranded tile (SST) nanostructure.

In some embodiments, the plurality of antigens comprises ovalbumin.

In some embodiments, the plurality of antigens is covalently linked to the nanostructure. In some embodiments, the plurality of antigens is covalently linked to free amine groups of the nucleic acid nanostructure.

In some aspects, the present disclosure provides a method of inducing a Th1 polarized immune response in cells, the method comprising administering to a subject (e.g., a human subject) the nucleic acid nanostructure provided herein.

In some aspects, the present disclosure provides a method of inducing a Th1 polarized immune response in cells, the method comprising administering to a subject a nucleic acid nanostructure comprising a plurality of uniformly spaced adjuvant molecules and a plurality of antigen molecules.

In some embodiments, the volume of the tumor is reduced at least 2-fold, at least 3-fold, at least 4-fold, at least 5-fold, relative to control (e.g., wherein the control is free CpG+free antigen+free nanostructure, or wherein the control is buffer only). In some embodiments, the volume of the tumor is reduced 2-fold, 3-fold, 4-fold, or 5-fold relative to control.

In some embodiments, the nucleic acid nanostructure is administered to the subject multiple times (e.g., at least 2 times, at least 3 times, etc.).

### Nucleic Acid Nanostructures

A “nucleic acid nanostructure,” as used herein, refers to nucleic acids that form (e.g., self-assemble) two-dimensional (2D) or three-dimensional (3D) shapes (e.g., reviewed in W. M. Shih, C. Lin, Curr. Opin. Struct. Biol. 20, 276 (2010), incorporated by reference herein). Nanostructures may be formed using any nucleic acid folding or hybridization methodology. One such methodology is DNA origami (see, e.g., Rothmund, P. W. K. Nature 440 (7082): 297-302 (2006), incorporated by reference herein). In a DNA origami approach, a nanostructure is produced by the folding of a longer “scaffold” nucleic acid strand through its hybridization to a plurality of shorter “staple” oligonucleotides, each of which hybridize to two or more non-contiguous regions within the scaffold strand. In some embodiments, a scaffold strand is at least 100 nucleotides in length. In some embodiments, a scaffold strand is at least 500, at least 1000, at least 2000, at least 3000, at least 4000, at least 5000, at least 6000, at least 7000, or at least 8000 nucleotides in length. The scaffold strand may be naturally or non-naturally occurring. Staple strands are typically less than 100 nucleotides in length; however, they may be longer or shorter depending on the application and depending upon the length of the scaffold strand. In some embodiments, a staple strand may be 15 to 100 nucleotides in length. In some embodiments, a staple strand is 25 to 50 nucleotides in length.

In some embodiments, a nucleic acid nanostructure is assembled in the absence of a scaffold strand (e.g., a scaffold-free structure). For example, a number of oligonucleotides (e.g., less than 200 nucleotides or less than 100 nucleotides in length) may be assembled to form a nucleic acid nanostructure.

Other methods for assembling nucleic acid nanostructures are known in the art, any one of which may be used herein. Such methods are described by, for example, Bellot G. et al., Nature Methods, 8:192-194 (2011); Liedl T. et al, Nature Nanotechnology, 5:520-524 (2010); Shih W. M. et al, Curr. Opin. Struct. Biol., 20:276-282 (2010); Ke Y. et al, J. Am. Chem. Soc, 131:15903-08 (2009); Dietz H. et al, Science, 325:725-30 (2009); Hogberg B. et al, J. Am. Chem. Soc, 131:9154-55 (2009); Douglas S. M. et al, Nature, 459:414-418 (2009); Jungmann R. et al, J. Am. Chem. Soc, 130:10062-63 (2008); Shih W. M., Nature Materials, 7:98-100 (2008); and Shih W. M., Nature, All: 618-21 (2004), each of which is incorporated herein by reference in its entirety.

A nucleic acid nanostructure may be assembled into one of many defined and predetermined shapes including without limitation a capsule, hemi-sphere, a cube, a cuboidal, a tetrahedron, a cylinder, a cone, an octahedron, a prism, a sphere, a pyramid, a dodecahedron, a tube, an irregular shape, and an abstract shape. The nanostructure may have a void volume (e.g., it may be partially or wholly hollow). In some embodiments, the void volume may be at least 25%, at least 50%, at least 75%, at least 85%, at least 90%, or more of the volume of the nanostructure. Thus, in some embodiments, nucleic acid nanostructures do not comprise a solid core. In some embodiments, nucleic acid nanostructures are not circular or near circular in shape. In some embodiments, nucleic acid nanostructures are not a solid core sphere. Depending on the intended use, nucleic acid nanostructures may be assembled into a shape as simple as a two-dimensional sheet or as complex as a three-dimensional capsule or lattice (or even more complex).

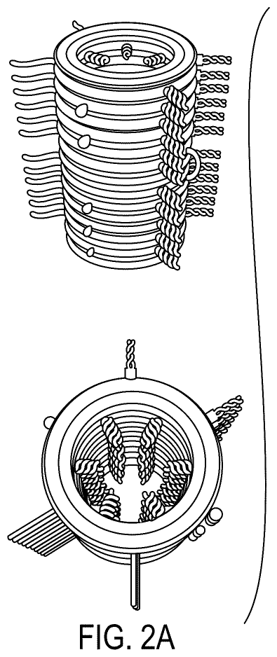

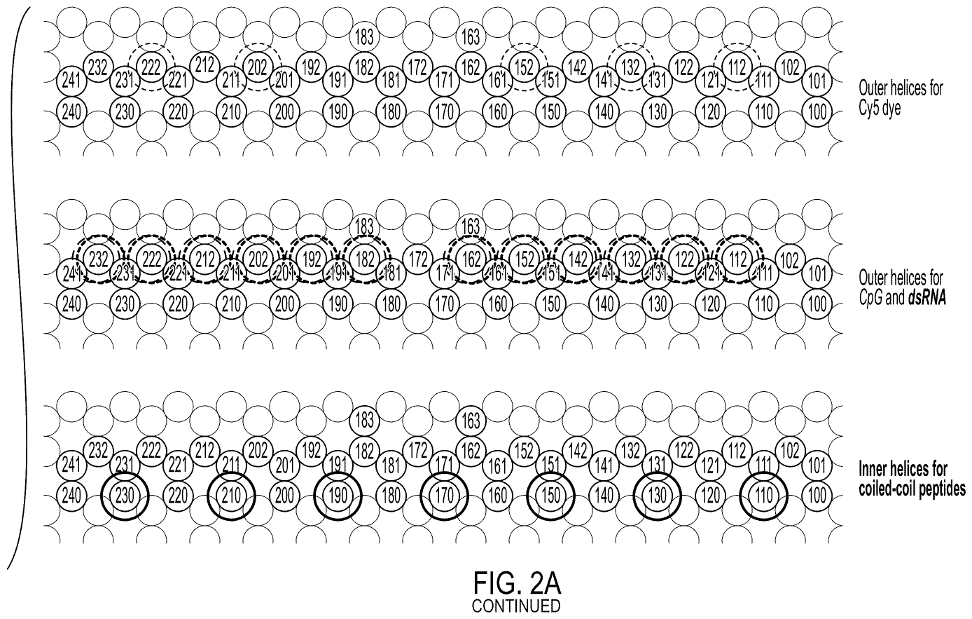

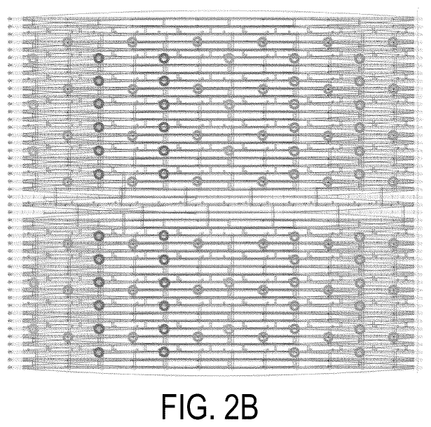

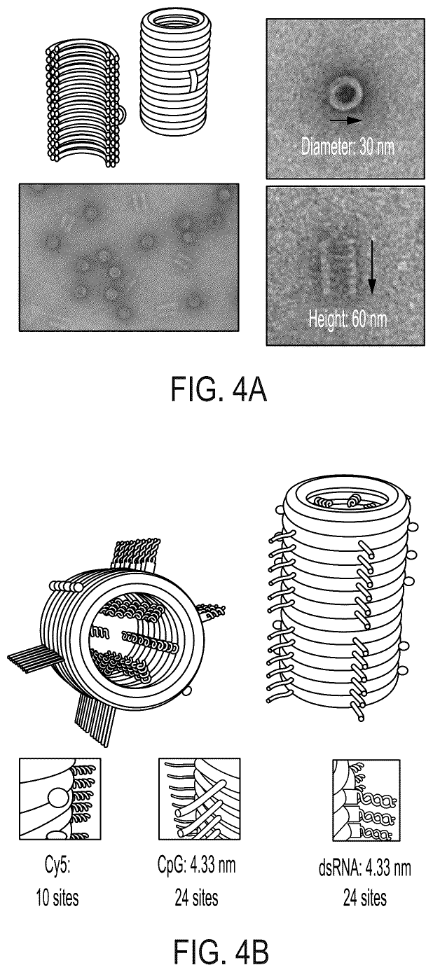

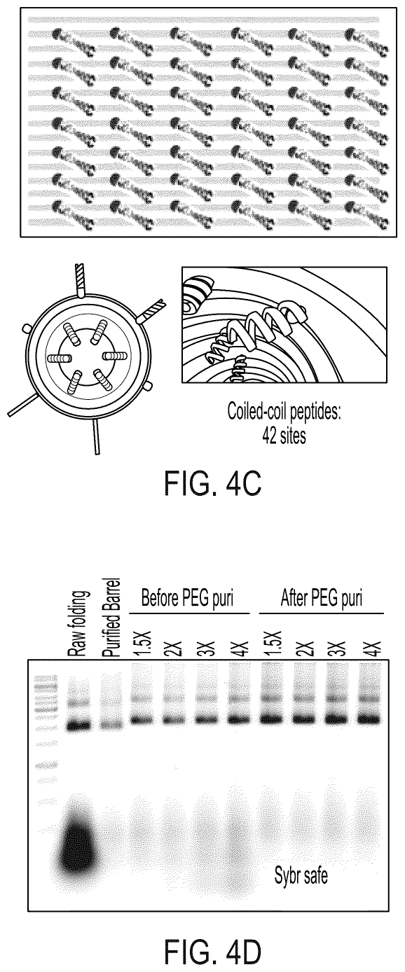

In some embodiments, the nucleic acid nanostructure comprises a barrel structure. See, e.g., FIGS. 2A-2B. A description of a three-dimensional DNA barrel structure is described in Wickham et al., Complex multicomponent patterns rendered on a 3D DNA-barrel pegboard. Nature Comm. (2020) 11:5768, incorporated herein in its entirety. In some embodiments, the DNA barrel has a diameter of 30 to 120 nm. As used herein, the diameter of a DNA barrel is measured from the helix mid-point to the outward-facing side of the nucleic acid (e.g., DNA). For example, the DNA barrel may have a diameter of 30 nm (see, e.g., FIG. 4A), 60 nm, or 90 nm. In some embodiments, the diameter of the DNA barrel is between 30 nm and 120 nm, between 40 nm and 110 nm, between 50 nm and 100 nm, between 60 nm and 90 nm, or between 70 nm and 80 nm. In some embodiments, the diameter of the DNA barrel is 30, 35, 40, 45, 50, 55, 60, 65, 70, 75, 80, 85, 90, 95, 100, 105, 110, 115, or 120 nm.

In some embodiments, the length of the DNA barrel is 15 to 250 nm. As described herein, the height of a DNA barrel is measured along the Y-axis of the cylindrical nanostructure as shown in FIG. 4A. For example, the height of the DNA barrel may be between 15 nm and 250 nm, between 20 nm and 240 nm, between 30 nm and 230 nm, between 40 nm and 220 nm, between 50 nm and 210 nm, between 60 nm and 200 nm, between 70 nm and 190 nm, between 80 nm and 180 nm, between 90 nm and 170 nm, between 100 nm and 160 nm, between 110 nm and 150 nm, between 120 nm and 140 nm, or between 125 nm and 135 nm. In some embodiments, the height of the DNA barrel is 15, 20, 30, 40, 50, 60, 70, 80, 90, 100, 110, 120, 130, 140, 150, 160, 170, 180, 190, 200, 210, 220, 230, or 240 nm.

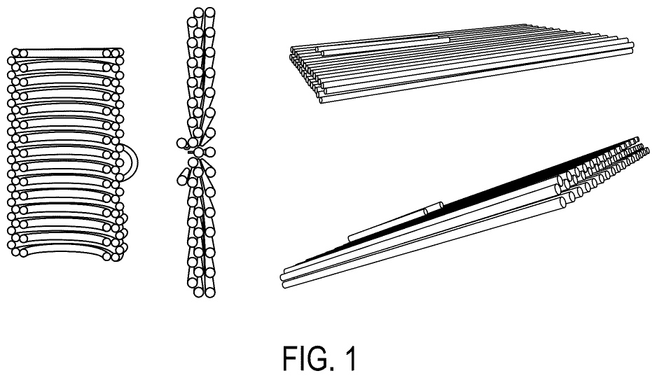

The barrel structure as described herein contains an exterior (i.e., outer) surface and interior (i.e., inner) surface. As used herein, the exterior surface of the DNA barrel refers to the area of the nanostructure that faces the convex region of the structure. The interior surface of the DNA barrel refers to the area of the nanostructure that faces the concave region of the structure (see, e.g., FIG. 1, left; FIG. 4A, top left; FIG. 4C). A useful feature of the DNA barrel is the ability to control the physical availability of molecules that are attached to and/or incorporated within the nanostructure. For example, CCPs located on the inner surface of the DNA barrel may be shielded from interaction with other objects, whereas adjuvants (e.g., CpG and/or dsRNA) and/or antigens located on the exterior surface of the DNA barrel are exposed to the environment, allowing them to interact with other objects, such as molecules, cells, nucleic acids, etc. In some embodiments, CCPs are located on the inner surface of the DNA barrel, and adjuvants (e.g., CpG and/or dsRNA) and/or antigens are located on the exterior surface of the DNA barrel.

Methods of assembling DNA barrel structures are known in the art, any one of which may be used herein. In some embodiments, the assembly of DNA barrels comprises mixing scaffold strands with tenfold excess of staple strands in a folding buffer comprising 5 mM Tris, 1 mM EDTA, and 6-20 mM MgCl2 prior to annealing the samples. In some embodiments, samples may be annealed using the following annealing ramps: 65-25 °C for 18 to 72 hours, 65 °C for 15 minutes and 50-40 °C for 18 to 72 hours, or 65 °C for 15 minutes and 47 °C for 18 to 72 hours. For example, the folding buffer may comprise 8 mM MgCl2, and the sample may be annealed at 47 °C for 18 hours. In other embodiments, the folding buffer may comprise 10 mM MgCl2 and may be annealed at a temperature of 65 °C for 15 minutes, which then decreases linearly from 50 to 40 °C for either 66 or 72 hours. In some embodiments, the folding buffer comprises 6, 7, 8, 9, 10, 11, 12, 13, 14, 15, 16, 17, 18, 19, or 20 mM MgCl2.

In some embodiments, DNA barrel structures are purified by methods known in the art. For example, DNA barrels may be purified by rate-zonal centrifugation using a 15-45% (v/v) glycerol gradient, wherein the glycerol solutions are made in TE buffer with 10 mM MgCl2, and spun at 40,000 to 55,000 rpm at 4 °C for 25 minutes to 1 hour. In some embodiments, DNA barrel structures are purified by PEG precipitation. For example, DNA barrels may be purified by mixing with 10%-20% PEG solution (e.g., 10%, 11%, 12%, 13%, 14%, 15%, 16%, 17%, 18%, 19%, or 20% PEG) at 1 to 1 ratio, wherein the PEG buffer are made in TE buffer with salt (e.g., 12 mM MgCl2, 510 mM NaCl₂), and subsequent centrifugation (e.g., spun at 16,000 rcf at 22 °C for 25 minutes). In some embodiments, DNA barrels are spun at 41,000 rpm at 4 °C for 25 minutes. In other embodiments, DNA barrels are spun at 55,000 rpm at 4 °C for 1 hour. In some embodiments, DNA barrels are spun at 40,000, 42,500, 45,000, 47,500, 50,000, 52,500, or 55,000 rpm. In some embodiments, DNA barrels are spun for 25, 30, 35, 40, 45, 50, 55, or 60 minutes.

In some embodiments, the nucleic acid nanostructure comprises a two- or three-dimensional square-lattice structure. A description of three-dimensional square-lattice structure is described in Yonggang Ke et al., Multilayer DNA Origami Packed on a Square Lattice. J Am Chem Soc. 2009 Nov. 4; 131 (43): 15903-8, incorporated herein in its entirety.

Nucleic acid nanostructures may be made of, or comprise, DNA, RNA, modified DNA, modified RNA, PNA, LNA or a combination thereof.

In some embodiments, nucleic acid nanostructures are rationally designed. A nucleic acid nanostructure is herein considered to be “rationally designed” if nucleic acids that form the nanostructure are selected based on pre-determined, predictable nucleotide base pairing interactions that direct nucleic acid hybridization. For example, nucleic acid nanostructures may be designed prior to their synthesis, and their size, shape, complexity and modification may be prescribed and controlled using certain select nucleotides (e.g., oligonucleotides) in the synthesis process. The location of each nucleic acid in the structure may be known and provided for before synthesizing a nanostructure of a particular shape. The fundamental principle for designing, for example, self-assembled nucleic acid nanostructures is that sequence complementarity in nucleic acid strands is selected such that, by pairing up complementary segments, the nucleic acid strands self-organize into a predefined nanostructure under appropriate physical conditions. Thus, in some embodiments, nucleic acid nanostructures are self-assembling. Similarly, handles and anti-handle nucleic acids (e.g., those linked to adjuvant and/or antigen) may be rationally designed to attach specifically to an interior or exterior surface of a nanostructure, in some embodiments, without intercalation or hybridization with nucleic acids forming the body of the nanostructure.

Examples of nucleic acid nanostructures for use in accordance with the present disclosure include, without limitation, capsules, lattices (E. Winfree, et al. Nature 394, 539 (1998); H. Yan, et al. Science 301, 1882 (2003); H. Yan, et al. Proc. Natl. Acad. of Sci. USA 100, 8103 (2003); D. Liu, et al. J. Am. Chem. Soc. 126, 2324 (2004); P. W. K. Rothemund, et al. PLOS Biology 2, 2041 (2004)), ribbons (S. H. Park, et al. Nano Lett. 5, 729 (2005); P. Yin, et al. Science 321, 824 (2008)), tubes (H. Yan Science (2003); P. Yin (2008)), finite two-dimensional (2D) and three dimensional (3D) objects with defined shapes (J. Chen, N. C. Seeman, Nature 350, 631 (1991); P. W. K. Rothemund, Nature 440, 297 (2006); Y. He, et al. Nature 452, 198 (2008); Y. Ke, et al. Nano. Lett. 9, 2445 (2009); S. M. Douglas, et al. Nature 459, 414 (2009); H. Dietz, et al. Science 325, 725 (2009); E. S. Andersen, et al. Nature 459, 73 (2009); T. Liedl, et al. Nature Nanotech. 5, 520 (2010); D. Han, et al. Science 332, 342 (2011)), and macroscopic crystals (J. P. Meng, et al. Nature 461, 74 (2009)). Other nucleic acid nanostructures may be used as provided herein.

Cadnano software may be used to design particular nucleic acid nanostructures of interest (see cadnano.org).

As used herein, the terms “nucleic acid” and/or “oligonucleotide” may refer to at least two nucleotides covalently linked together. A nucleic acid of the present disclosure may generally contain phosphodiester bonds, although in some cases, nucleic acid analogs are included that may have other backbones, comprising, for example, phosphoramide (Beaucage et al., Tetrahedron 49 (10): 1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 58”, “Carbohydrate Modifications in Antisense Resea” ch”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 58”, “Carbohydrate Modifications in Antisense Resea” ch”, Ed. Y. S. Sanghui and P. Dan Cook. Nucleic acids containing one or more carbocyclic sugars are also included within the definition of nucleic acids (see Jenkins et al., Chem. Soc. Rev. (1995) pp 169-176). Several nucleic acid analogs are described in Rawls, C & E News Jun. 2, 1997 page 35. All of these references are hereby expressly incorporated by reference. Nucleic acid may have a homogenous backbone (e.g., entirely phosphodiester or entirely phosphorothioate) or a heterogeneous (or chimeric) backbone. Phosphorothioate backbone modifications render a nucleic acid less susceptible to nucleases and thus more stable (as compared to a native phosphodiester backbone nucleic acid) under certain conditions. Other linkages that may provide more stability to a nucleic acid include without limitation phosphorodithioate linkages, methylphosphonate linkages, methylphosphorothioate linkages, boranophosphonate linkages, peptide linkages, alkyl linkages, dephospho type linkages, and the like. Thus, in some instances, nucleic acids have non-naturally occurring backbones. Modifications of the ribose-phosphate backbone may be done, for example, to facilitate the addition of labels, or to increase the stability and half-life of such molecules in physiological environments.

Nucleic acids may be single-stranded (ss) or double-stranded (ds), as specified, or may contain portions of both single-stranded and double-stranded sequence (e.g., are partially double-stranded). Nucleic acids may be DNA, both genomic and cDNA, RNA or a hybrid, where the nucleic acid contains any combination of deoxyribo- and ribonucleotides, and any combination of bases, including uracil, adenine, thymine, cytosine, guanine, inosine, xathanine hypoxathanine, isocytosine, and isoguanine. As used herein, the term “nucleoside” includes nucleotides as well as nucleoside and nucleotide analogs, and modified nucleosides such as amino modified nucleosides. In addition, “nucleoside” includes non-naturally occurring analog structures. Thus, for example, the individual units of a peptide nucleic acid, each containing a base, are referred to herein as a nucleoside.

Nucleic acids include DNA such as B-form DNA, D-form DNA and L-form DNA and RNA, as well as various modifications thereof. Modifications include base modifications, sugar modifications, and backbone modifications. Non-limiting examples of these are provided below.

Non-limiting examples of DNA variants that may be used as provided herein are L-DNA (the backbone enantiomer of DNA, known in the literature), peptide nucleic acids (PNA) bisPNA clamp, a pseudocomplementary PNA, a locked nucleic acid (LNA), or co-nucleic acids of the above such as DNA-LNA co-nucleic acids. It is to be understood that nucleic acids used as provided herein may be homogeneous or heterogeneous in nature. As an example, they may be completely DNA in nature or they may comprise DNA and non-DNA (e.g., LNA) monomers or sequences. Thus, any combination of nucleic acid elements may be used. The nucleic acid modification may render the nucleic acid more stable and/or less susceptible to degradation under certain conditions. For example, in some instances, the nucleic acids are nuclease-resistant.

Methods of synthesizing nucleic acids (e.g., ssDNA or dsDNA, or ssRNA or dsRNA) are known in the art and are described, for example, in U.S. Pat. Nos. 5,143,854 and 5,445,934, herein incorporated in their entirety.

Nucleic acids may be synthesized in vitro. Methods for synthesizing nucleic acids, including automated nucleic acid synthesis, are also known in the art. Nucleic acids having modified backbones, such as backbones comprising phosphorothioate linkages, and including those comprising chimeric modified backbones may be synthesized using automated techniques employing either phosphoramidate or H-phosphonate chemistries. (F. E. Eckstei”, “Oligonucleotides and Analogu-s-A Practical Appro” ch” IRL Press, Oxford, U K, 1991, and M. D. Matteucci and M. H. Caruthers, Tetrahedron Lett. 21, 719 (1980)) Aryl- and alkyl-phosphonate linkages can be made, e.g., as described in U.S. Pat. No. 4,469,863; and alkylphosphotriester linkages (in which the charged oxygen moiety is alkylated), e.g., as described in U.S. Pat. No. 5,023,243 and European Patent No. 092,574, can be prepared by automated solid phase synthesis using commercially available reagents. Methods for making other DNA backbone modifications and substitutions have been described. Uhlmann E et al. (1990) Chem Rev 90:544; Goodchild J (1990) Bioconjugate Chem 1:165; Crooke S T et al. (1996) Annu Rev Pharmacol Toxicol 36:107-129; and Hunziker J et al. (1995) Mod Synth Methods 7:331-417.

Nucleic acids may additionally or alternatively comprise modifications in their sugars. For example, a β-ribose unit or a β-′-2′-deoxyribose unit can be replaced by a modified sugar unit, wherein the modified sugar unit is for example selected from β-D-ribose, α-′-2′-deoxyribose, ′-2′-deoxyribose′ 2′-′-2′-deoxyribose, arabinose′ 2′-F-arabinose′ 2′-O—(C₁-C₆)alkyl-ribose, preferabl’ 2′-O-(C₁-C₆)alkyl-ribose i′ 2′-O-methylribose′ 2′-O—(C₂-C₆)alkenyl-ribose′ 2′-[O-(C₁-C₆)alkyl-O-(C₁-C₆)alkyl]-ribose′ 2′-NH-2′-deoxyribose, β-D-xylo-furanose, α-arabinofuranose, 2,4-dideoxy-β-D-erythro-hexo-pyranose, and carbocyclic (described, for example, in Froehler J (1992) Am Chem Soc 114:8320) and/or open-chain sugar analogs (described, for example, in Vandendriessche et al. (1993) Tetrahedron 49:7223) and/or bicyclosugar analogs (described, for example, in Tarkov M et al. (1993) Helv Chim Acta 76:481).

Nucleic acids may comprise modifications in their bases. Modified bases include modified cytosines (such as 5-substituted cytosines (e.g., 5-methyl-cytosine, 5-fluoro-cytosine, 5-chloro-cytosine, 5-bromo-cytosine, 5-iodo-cytosine, 5-hydroxy-cytosine, 5-hydroxymethyl-cytosine, 5-difluoromethyl-cytosine, and unsubstituted or substituted 5-alkynyl-cytosine), 6-substituted cytosines, N4-substituted cytosines (e.g., N4-ethyl-cytosine), 5-aza-cytosine, 2-mercapto-cytosine, isocytosine, pseudo-isocytosine, cytosine analogs with condensed ring systems (e.g., N,N′-propylene cytosine or phenoxazine), and uracil and its derivatives (e.g., 5-fluoro-uracil, 5-bromo-uracil, 5-bromovinyl-uracil, 4-thio-uracil, 5-hydroxy-uracil, 5-propynyl-uracil), modified guanines such as 7-deazaguanine, 7-deaza-7-substituted guanine (such as 7-deaza-7-(C2-C₆)alkynylguanine), 7-deaza-8-substituted guanine, hypoxanthine, N2-substituted guanines (e.g. N2-methyl-guanine), 5-amino-3-methyl-3H,6H-thiazolo[4,5-d]pyrimidine-2,7-dione, 2,6-diaminopurine, 2-aminopurine, purine, indole, adenine, substituted adenines (e.g. N6-methyl-adenine, 8-oxo-adenine) 8-substituted guanine (e.g. 8-hydroxyguanine and 8-bromoguanine), and 6-thioguanine. The nucleic acids may comprise universal bases (e.g. 3-nitropyrrole, P-base, 4-methyl-indole, 5-nitro-indole, and K-base) and/or aromatic ring systems (e.g. fluorobenzene, difluorobenzene, benzimidazole or dichloro-benzimidazole, 1-methyl-1H-[1,2,4]triazole-3-carboxylic acid amide). A particular base pair that may be incorporated into the oligonucleotides of the invention is a dZ and dP non-standard nucleobase pair reported by Yang et al. NAR, 2006, 34 (21): 6095-6101. dZ, the pyrimidine analog, is 6-amino-5-nitro-3-(1′-β-D-2′-deoxyribofuranosyl)-2 (1H)-pyridone, and its Watson-Crick complement dP, the purine analog, is 2-amino-8-(1′-β-D-1′-deoxyribofuranosyl)-imidazo[1,2-a]-1,3,5-triazin-4 (8H)-one.

In exemplary embodiments, nucleic acid nanostructures comprise single-stranded genomic DNA. For example, nucleic acid nanostructures may comprise linear or circular single-stranded M13 plasmid DNA. In some embodiments, nucleic acid nanostructures do not comprise plasmid DNA.

It should be appreciated that nucleic acid nanostructures of the present disclosure, in some embodiments, do not include condensed nucleic acid. As used herein, “condensed nucleic acid” refers to compacted nucleic acid, for example, that is twisted and coiled upon itself (see, e.g., Teif V B, et al. Progress in Biophysics and Molecular Biology 105 (3): 208-222, incorporated by reference herein). The term “condensed nucleic acid” excludes nucleic acid nanostructures that have a distinct 2D or 3D architecture.

It should also be appreciated that nucleic acid nanostructures of the present disclosure, in some embodiments, do not include coding nucleic acid. That is, in some embodiments, nucleic acid nanostructures comprise non-coding nucleic acids (e.g., nucleic acids that do not encode proteins). As used herein, a “coding nucleic acid” refers to a nucleic acid containing a nucleotide sequence that specifies a sequence of amino acids of a protein (e.g., a therapeutic protein). Thus, a “non-coding nucleic acid” is a nucleic acid that does not specify a sequence of amino acids of a protein and, accordingly, is not transcribed into RNA or translated into protein. In other embodiments, it should be understood that a nucleic acid nanostructure may contain one or more coding nucleic acids.

In some embodiments, nucleic acids used to make nucleic acid nanostructures do not code for any amino acid. In some embodiments, nucleic acids used to make nucleic acid nanostructures do not code for more than 1, 2, 3, 4 or 5 consecutive amino acids.

In some embodiments, nucleic acids used to make nucleic acid nanostructures do not include art-recognized regulatory elements/sequences such as promoters, enhancers, polyA sequences and/or ribosomal binding site sequences.

In some embodiments, nucleic acids used to make nucleic acid nanostructures are not plasmids.

In some embodiments, nucleic acids used to make nucleic acid nanostructures contain more than one nucleic acid, and the nucleic acid are different from each other. That is, the nucleic acids of a nucleic acid nanostructure may comprise a plurality of different nucleic acids.

In some embodiments, nucleic acid nanostructures are not encapsulated by or coated with (e.g., linked to) lipids. For example, a variety of gene delivery methods of the prior art make use of nucleic acid nanostructures that are linked to hydrophobic moieties and/or covered by lipids (e.g., such as a lipid bilayer), which function to prevent nuclease degradation (see, e.g., WO 2013148186 A1). The present disclosure, in some embodiments, excludes nucleic acid nanostructures that are linked to hydrophobic moieties and/or covered by lipids. In other embodiments, however, a nucleic acid nanostructure may contain one or more nucleic acids linked to one or more hydrophobic moieties and/or lipids.

Nucleic acid nanostructures of the present disclosure have a variety of in vitro and in vivo uses. In some embodiments, nucleic acid nanostructures are used as scaffolds, cages or multifunctional carriers for delivering an antigen that is intended for use in vivo and/or in vitro. A nucleic acid nanostructure may be delivered by any suitable delivery method, for example, intravenously or orally.

The present disclosure contemplates imparting addressability to nucleic acid nanostructures. For example, nucleic acid nanostructures may be modified by site-specific attachment of targeting moieties such as proteins, ligands or other small biomolecules. In some embodiments, nucleic acid nanostructures may comprise nucleic acid “staple” strands, as described above, that serve as handles for nanometer-specific placement of accessory molecules (e.g., biotin/streptavidin) at virtually any position on or within the structure (see, e.g., Stein et al. Chemphyschem. 12 (3), 689-695 (2011); Steinhauer et al. Angew Chem. Int. Ed. Engl. 48 (47), 8870-8873 (2009); Stein et al. J. Am. Chem. Soc. 133 (12), 4193-4195 (2011); Kuzyk et al. Nature 483 (7389), 311-314 (2012); and Ding et al. J. Am. Chem. Soc. 132 (10), 3248-3249 (2010); Yan et al. Science 301 (5641), 1882-1884 (2003); and Kuzuya et al. Chembiochem. 10 (11), 1811-1815 (2009), each of which is incorporated by reference herein).

In some embodiments, nucleic acids of nanostructures provided herein are modified (e.g., covalently modified) with a linker (e.g., biotin linker) during synthesis or via enzymatic means (see, e.g., Jahn et al. Bioconjug. Chem. 22 (4), 819-823 (2011) incorporated by reference herein). Such methods may also be used to position reaction systems on nucleic acid nanostructures through the chemical biotinylation of enzyme molecules (see, e.g., Voigt et al. Nat. Nanotechnol. 5 (3), 200-203 (2010)).

A more generalized antibody-based binding approach may also be used to link target proteins to nucleic acid nanostructures at defined distances (see, e.g., Williams et al. Angew Chem. Int. Ed. Engl. 46 (17), 3051-3054 (2007); and He Y et al. J. Am. Chem. Soc. 128 (39), 12664-12665 (2006), each of which is incorporated by reference herein). Thus, in some embodiments, nucleic acid nanostructures are linked to one or more antibodies.

In other embodiments, DNA aptamers, which adopt a specific secondary structure with high binding affinity for a particular molecular target, are used as linkers, thereby eliminating the need for protein linkers (see, e.g., Ellington et al. Nature 346 (6287), 818-822 (1990); Chhabra et al. J. Am. Chem. Soc. 129 (34), 10304-10305 (2007); and Rinker et al. Nat. Nanotechnol. 3 (7), 418-422 (2008), each of which is incorporated by reference herein).

The present disclosure also contemplates the use of recombinant genetic engineering methods to selectively add affinity tags or other peptide linkers to nucleic acid nanostructures. For example, polyhistidine sequence consisting of multiple histidine residues on the C- or N-terminus end of a target protein is a commonly used tag for affinity-based purification. This, in turn, can be linked via nickel-mediated interaction to a nitrilotriacetic acid molecule that is covalently conjugated to an amine (see, e.g., Goodman et al. Chembiochem. 10 (9), 1551-1557 (2009), incorporated by reference herein) or thiol-modified (see, e.g., Shen et al. J. Am. Chem. Soc. 131 (19), 6660-6661 (2009), incorporated by reference herein) nucleic acid. Through this method, fluorescent proteins may be positioned both periodically and specifically on nucleic acid nanostructures (Goodman et al. (2009); and Shen et al. (2009)). Similarly, SNAP and HaloTag® peptide sequences, also used for affinity purification of recombinant proteins, may be utilized for the orthogonal decoration of nucleic acid nanostructures with different protein or enzyme species (see, e.g., Sacca et al. Angew Chem. Int. Ed. Engl. 49 (49), 9378-9383 (2010), incorporated by reference herein). A related approach involving the creation of chimeric proteins conjugated to a DNA-binding domain, can eliminate the often complex chemical synthesis techniques and toxic compounds (e.g., nickel) necessary to stably conjugate affinity tag binding partners to oligonucleotide strands. Further, zinc-finger domains that recognize specific double-stranded sequences may be used to arrange fluorescent proteins at specific locations on nucleic acid nanostructures of the present disclosure (see, e.g., Nakata et al. Angew Chem. Int. Ed. Engl. 51 (10), 2421-2424 (2012), incorporated by reference herein).

An adjuvant (e.g., CpG and/or dsRNA) and/or antigen may be covalently or non-covalently attached to a nucleic acid nanostructure. The location and nature of the linkage between the adjuvant (e.g., CpG and/or dsRNA) and/or antigen and the nucleic acid nanostructure will depend upon the function of the adjuvant (e.g., CpG and/or dsRNA) and/or antigen. As an example, an adjuvant (e.g., CpG and/or dsRNA) and/or antigen may be intended to release (including slow release) from the nanostructure, and in that case, the linkage between the adjuvant (e.g., CpG and/or dsRNA) and/or antigen and the nanostructure may be chosen to achieve the desired release profile. In some embodiments, an adjuvant (e.g., CpG and/or dsRNA) and/or antigen is inactive in its bound form and activated only when released.

In some embodiments, an adjuvant (e.g., CpG and/or dsRNA) and/or antigen is combined with nucleic acids during assembly (e.g., self-assembly) of nanostructures, or an adjuvant (e.g., CpG and/or dsRNA) and/or antigen is combined with pre-formed nucleic acid nanostructures.

An adjuvant (e.g., CpG and/or dsRNA) and/or antigen may be linked to an interior surface (in the interior compartment) or an exterior surface of a nanostructure. An adjuvant (e.g., CpG and/or dsRNA) and/or antigen may be arranged in various configurations. In some embodiments, an adjuvant (e.g., CpG and/or dsRNA) and/or antigen is indirectly linked to the nucleic acid nanostructure through a handle/anti-handle hybridization system. The term “handle”, as used herein, refers to an extension of staple strands within the nucleic acid nanostructure. The term “anti-handle”, as used herein, refers to a nucleic acid sequence complementary to a nucleic acid handle (i.e., handles and anti-handles can bind one another). In some embodiments, the handle and/or anti-handle is a double-stranded DNA molecule, a single-stranded DNA molecule, a single-stranded RNA molecule, or a double-stranded RNA molecule. In some embodiments, the handle and/or anti-handle comprises nucleotides that have been modified (e.g., Pseudouridine-5′-Triphosphate, 5-Methoxyuridine-5′-Triphosphate, N1-Methylpseudouridine-5′-Triphosphate, etc.). The length of handles and anti-handles may vary. For example, handles and anti-handles may be 5 to 50 nucleotides in length. In some embodiments, handles and anti-handles are 5, 10, 15, 20, 25, 30, 35, 40, 45, or 50 nucleotides in length. In some embodiments, anti-handles may be chemically conjugated to an adjuvant (i.e., CpG and/or dsRNA) and/or antigen, so that upon hybridization (i.e., complementary binding) of handles to anti-handles, the adjuvant (e.g., CpG and/or dsRNA) and/or antigen become indirectly linked to nucleic acid nanostructures. It should be understood that nanostructures of the present disclosure permit precise placement of an adjuvant (e.g., CpG and/or dsRNA) and/or antigen or more than one adjuvant (e.g., CpG and/or dsRNA) and/or antigen (e.g., a combination of different adjuvant (e.g., CpG and/or dsRNA) and/or antigen) on the interior and/or exterior surface of the nanostructures.

Nucleic acid nanostructures of the present disclosure permit high-density “packing” of adjuvant (e.g., CpG and/or dsRNA) and/or antigen on and into the nanostructures. In some embodiments, a nucleic acid nanostructure is decorated with one adjuvant (e.g., CpG or dsRNA) and/or antigen per 50 nm² to 75 nm². In some embodiments, a nucleic acid nanostructure is decorated with one adjuvant (e.g., CpG or dsRNA) and/or antigen per 50 nm², 55 nm², 60 nm², 65 nm², 70 nm² or 75 nm². For example, using a rhombic-lattice spacing for a 30 nm tall, 60 nm diameter cylindrical nanostructure, 72 positions on the exterior of the nanostructure and 84 positions on the interior may be occupied by adjuvant (e.g., CpG and/or dsRNA) and/or antigen. For larger nanostructures, for example, those with two 30 nm×60 nm cylindrical nanostructures, the number of positions occupied by adjuvant (e.g., CpG and/or dsRNA) and/or antigen is doubled. For even larger nanostructures, for example, those with three 30 nm×60 nm cylindrical nanostructures, the number of positions occupied by adjuvant (e.g., CpG and/or dsRNA) and/or antigen tripled, and so on.

The present disclosure contemplates, in some aspects, the delivery of nucleic acid nanostructures, or nucleic acid nanostructures loaded with an adjuvant (e.g., CpG and/or dsRNA) and/or antigen, systemically or to localized regions, tissues or cells. Any adjuvant (e.g., CpG and/or dsRNA) and/or antigen gent may be delivered using the methods of the present disclosure provided that it can be loaded onto or into the nucleic acid nanostructure. Because such processes are relatively innocuous, it is expected that virtually any adjuvant (e.g., CpG and/or dsRNA) and/or antigen may be used.

The length of a CpG adjuvant may vary. For example, the length may be 10-100 nucleotides (nt), 10-50 nt, or 10-20 nt.

### Adjuvants

An “adjuvant” is an agent that enhances an immune response to an antigen. In some embodiments, an adjuvant is a CpG oligonucleotide. CpG oligonucleotides are short single-stranded synthetic DNA molecules that contain a cytosine triphosphate deoxynucleotide (“C”) followed by a guanine triphosphate deoxynucleotide (“G”). The “p” refers to the phosphodiester, or modified phosphorothioate (PS), linkage between consecutive nucleotides. CpG oligonucleotides typically enhance the immunostimulatory effect of nucleic acid nanostructures (Li, J. et al. ACS NANO, 5 (11): 8783-8789, 2011; Schuller, V. et al. ACS NANO, 5 (12): 9696-9702, 2011). For example, after they are taken up by cells, CpG oligonucleotides, which are a hallmark of microbial DNA, are recognized by the endosomal Toll-like receptor 9 (TLR9) that activates downstream pathways to induce immunostimulatory effects, producing high-level secretion of various pro-inflammatory cytokines including tumor necrosis factor (TNF)-α, interleukin (IL)-6, and IL-12. In some embodiments, CpG oligonucleotides are linked to an interior surface of a nucleic acid nanostructure. In some embodiments, CpG oligonucleotides are linked to an exterior surface of a nucleic acid nanostructure. In some embodiments, a nucleic acid nanostructure has CpG oligonucleotides linked to both an interior and exterior surface. Other examples of adjuvants include, without limitation, lipopolysaccharide and polyI:C (dsRNA mimic).

### Antigens

An “antigen”, as used herein, is any biomolecule that induces an immune response. In some embodiments, an antigen is a peptide, a protein or polypeptide, or a nucleic acid. In some embodiments, antigens include a mixture of proteins derived from tumor cells. In some embodiments, an antigen is a cancer antigen. A cancer antigen is a type of protein or molecule that is produced by cancer cells and can be detected by the immune system. A cancer antigen may be, for example, a component or element of a cancer cell or a biomolecule isolated from a cancer cell (e.g., a biomolecule known to be associated with cancerous tumors). In some embodiments, a cancer antigen comprises a biomolecule (e.g., a peptide or polypeptide) that is overexpressed or overactivated in cancer cells, relative to normal and non-cancerous cells. In some embodiments, an antigen is associated with an infectious disease. In some embodiments, an antigen associated with an infectious disease is a type of protein or molecule that is produced by a viral particle or cell associated with the infectious disease.

Non-limiting examples of cancer antigens include Her2 peptides (for vaccination against selected breast cancers); NY-ESO-1 peptides (for vaccination against selected bladder cancers); HPV16 E7 peptides (for vaccination against selected cervical cancers); carcinoembryonic antigen (for vaccination against selected colorectal cancers); Wilms' tumor 1 (WT1) peptides (for vaccination against selected leukemias); MART-1, gp100, and tyrosinase (for vaccination against selected melanomas); URLC10, VEGFR1, and VEGFR2 (for vaccination against selected non-small lung cell cancers); survivin (for vaccination against selected ovarian cancers); MUC1 (for vaccination against selected pancreatic cancers); MUC2 (for vaccination against selected prostate cancers); telomerase (TERT); Indoleamine 2,3-dioxygenase (IDO1); CTAG1B, and VEGF receptors (FLT1 and KDR). In some embodiments, a cancer antigen is as described in Tagliamonte, M. et al. “Antigen-specific vaccines for cancer treatment”, Hum Vaccin Immunother. 2014 November; 10 (11): 3332-3346.; or Pol, J. et al. “Trial Watch: Peptide-based anticancer vaccines”, Oncoimmunology. 2015 April; 4 (4): e974411.

In some embodiments, a cancer antigen is selected from the following: CEA; gp100; Pmell7; mammaglobin-A; Melan-A; MART-1; NY-BR-1; ERBB2; OA1; PAP; PSA; RAB38; NY-MEL-1; TRP-1; gp75; TRP-2; tyrosinase; WT1; CD33; BAGE-1; D393-CD20n; Cyclin-A1; GAGE-1,2,8; GAGE-3,4,5,6,7; GnTVf; HERV-K-MEL; KK-LC-1; KM-HN-1; LAGE-1; LY6K; MAGE-A1; MAGE-A2; MAGE-A3; MAGE-A4; MAGE-A6; MAGE-A9; MAGE-A10; MAGE-A12m; MAGE-C1; MAGE-C2; mucink; NA88-A; NY-ESO-1; LAGE-2; SAGE; Spl7; SSX-2; SSX-4; survivin; BIRC5; TAG-1; TAG-2; TRAG-3; TRP2-INT2g; XAGE-lb; GAGED2a; BCR-ABL (b3a2); adipophilin; AIM-2; ALDH1A1; BCLX (L); BING-4; CALCA; CD45; CD274; CPSF; cyclin DI; DKKI; ENAH (hMena); EpCAM; EphA3; EZH2; FGF5; glypican-3; G250; MN; CAIX; HER-2; neu; HLA-DOB; Hepsin; IDUA; IGF2B3; IL13Ralpha2; Intestinal carboxyl esterase; alpha-foetoprotein; Kallikrein 4; KIF20A; Lengsin; M-CSF; MCSP; mdm-2; Meloe; Midkine; MMP-2; MMP-7; MUC1; MUC5AC; p53; PAX5; PBF; PRAME; PSMA; RAGE-1; RGS5; RhoC; RNF43; RU2AS; secernin 1; SOX10; STEAP1; Telomerase; TPBG; and VEGF.

### Antigen-Capturing Motifs

An “antigen-capturing motif”, as used herein, refers to any biomolecule that binds to an antigen (e.g., a neoantigen). In some embodiments, the antigen-capturing motif is a nucleic acid (e.g., DNA and/or RNA), a peptide, and/or a protein. In some embodiments, the antigen-capturing motif is hydrophobic. In some embodiments, a hydrophobic antigen-capturing motif is capable of binding to and capturing hydrophobic immunogenic neoantigens. In some embodiments, the antigen-capturing motif is hydrophilic. In some embodiments, a hydrophilic antigen-capturing motifs is capable of binding to and capturing hydrophilic immunogenic neoantigens. In some embodiments, the antigen-capturing motif is uncharged, negatively charged, or positively charged.

In some embodiments, the antigen-capturing motif is attached via its N-terminus to the DNA nanostructure. In some embodiments, the antigen-capturing motif is attached via its C-terminus to the DNA nanostructure.

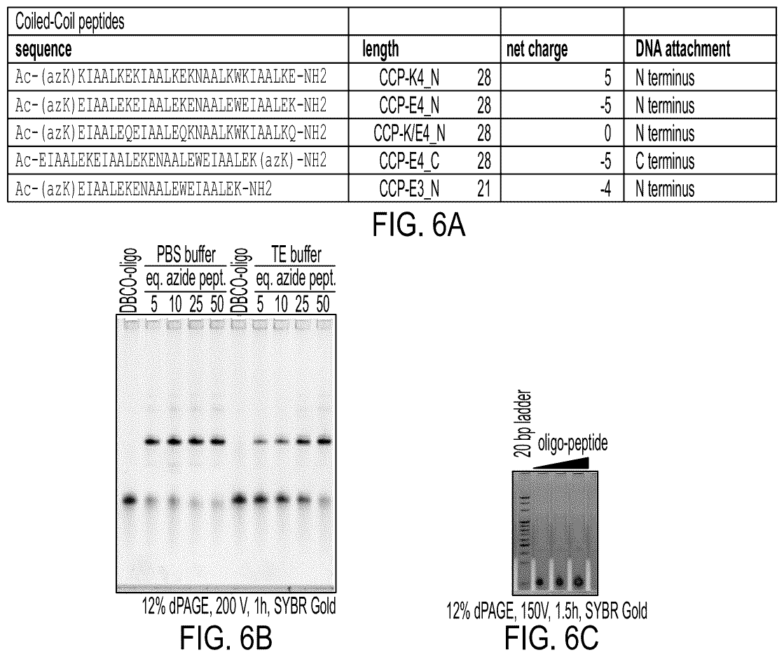

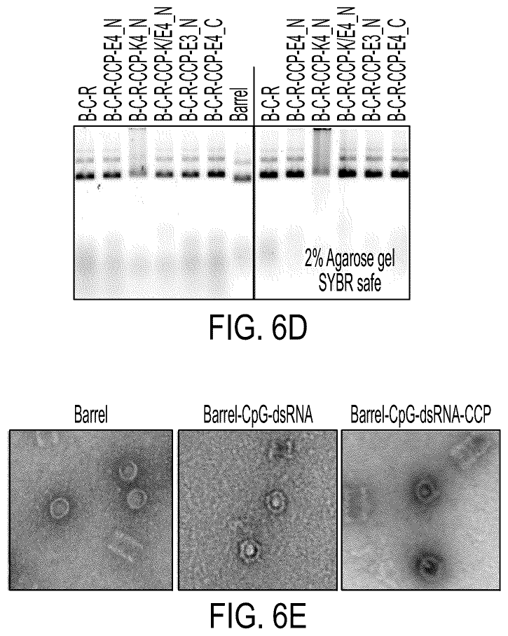

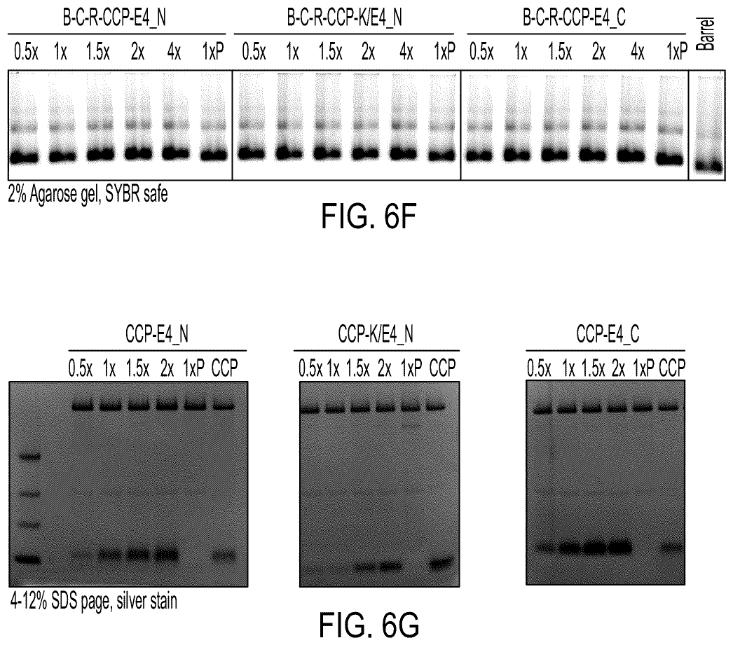

In some embodiments, the antigen-capturing motif is a coiled-coil peptide (see, e.g., FIG. 6A). A coiled-coil peptide (CCP) is a common structural motif consisting of two or more a-helices wrapped around each other into a superhelical bundle. There can be two, three or four helices in the bundle and they can run in the same (parallel) or in the opposite (antiparallel) directions. Examples of proteins comprising coiled-coil motifs include, but are not limited to, myosins, tropomyosins, intermediate filaments, keratin, fibrinogen, c-Fos, and c-Jun. The amino acid sequences of peptides forming coiled-coil bundles are characterized by a heptad repeating unit denoted as (abcdefg)ₙ, where n is the number of repeats. The interaction between helices reduces the typical α-helical pitch from 3.6 to 3.5 residues per turn, creating an interfacial stripe between associating helices, where residues in the a and d positions are typically hydrophobic. This forms the core of the coiled-coil via the packing of hydrophobic amino acids, and this core is stabilized by both hydrophobic and van der Waals interactions. Importantly, the type of hydrophobic amino acids present in the interface can specify the number of helices in each superhelix bundle. The e and g positions tend to be occupied by polar/charged amino acids, together creating complementary charge pairs across the bundle, and these help stabilize the coiled coil via inter-strand electrostatic interactions. Both the hydrophobic interactions arising from residues in the a and d positions and the electrostatic interactions between residues in the e and g positions can be utilized to influence the oligomerization state, parallel versus antiparallel topology, registry, and thermodynamic stability of bundles. Residues in the b, c, and f positions can be used to provide sufficient solubility of the peptides, as well as to control the higher-order aggregation of oligomers via the exterior surface of the coiled coil.

In some embodiments, the antigen-capturing motifs (e.g., CCPs) are 10-50 amino acids in length. In some embodiments, the antigen-capturing motifs (e.g., CCPs) are 15 to 45, 20 to 40, 25 to 35, or about 30 amino acids in length. In some embodiments, the antigen-capturing motifs (e.g., CCPs) are 10, 15, 20, 25, 30, 35, 40, 45, and/or 50 amino acids in length.

In some embodiments, the antigen-capturing motifs (e.g., CCPs) comprise modified amino acids (e.g., epsilon azido lysine (azK), 4-hydroxyproline, 5-hydroxylysine, 6-N-methylysine, gamma-carboxyglutamate, desmosine, selenocysteine, phosphoserine (pSer), phosphothreonine (pThr), phosphotyrosine (pTyr), sulfo-Tyrosine, Arg (Me) 2 symmetric, Arg (Me) 2 asymmetric, Arg (Me), argpyrimidine, Asn (GlcNAc), MeLys, (Me) 2Lys, (Me) 3Lys, acLys, carboxymethyllysine, or Thr (GalNAc).

In some embodiments, the CCPs attached to the DNA nanostructure provided herein have the following amino acid sequences:

| | | SEQ ID |
|---|---|---|
| Name | Sequence | NO: |
| K4_N | Ac-(azK)KIAALKEKIAALKEKNAAL | 1 |
| | KWKIAALKE-NH₂ | |
| K4_N | KKIAALKEKIAALKEKNAALKWKIAAL | 2 |
| (unmodified | KE | |
| sequence) | | |
| E4_N | Ac-(azK)EIAALEKEIAALEKENAAL | 3 |
| | EWEIAALEK-NH₂ | |
| E4_N | KEIAALEKEIAALEKENAALEWEIAAL | 4 |
| (sequence) | EK | |
| K/E4N | Ac-(azK)EIAALEQEIAALEQKNAAL | 5 |
| | KWKIAALKQ-NH₂ | |
| K/E4N | KEIAALEQEIAALEQKNAALKWKIAAL | 6 |
| (unmodified | KQ | |
| sequence) | | |
| E4_C | Ac-EIAALEKEIAALEKENAALEWEIA | 7 |
| | ALEK(azK)-NH₂ | |
| E4_C | EIAALEKEIAALEKENAALEWEIAALE | 8 |
| (unmodified | KK | |
| sequence) | | |
| E3_N | Ac-(azK)EIAALEKENAALEWEIAAL | 9 |
| | EK-NH₂ | |
| E3_N | KEIAALEKENAALEWEIAALEK | 10 |
| (unmodified | | |
| sequence) | | |

* “Ac” denotes an acetyl group; “NH₂” denotes an amine

### Methods of Use

The nucleic acid nanostructure vaccines of the disclosure may be useful in providing a therapeutic benefit (e.g., treatment) when administered to subjects. Nucleic acid nanostructure vaccines (e.g., a nucleic acid Barrel nanostructure conjugated to adjuvant molecules and antigen-capturing motifs) may be used to treat disease in subjects (e.g., human subjects). In some embodiments, nucleic acid nanostructure vaccines are used to treat cancer in subjects (e.g., human subjects). Accordingly, for example, in some embodiments, provided herein are methods of treating a subject having cancer by administering a nucleic acid nanostructure vaccine of the disclosure. In some embodiments, a subject is prophylactically administered a nucleic acid nanostructure vaccine (e.g., to treat cancer by preventing tumor formation or progression). In some embodiments, provided herein are methods of treating a subject having an infectious disease by administering a nucleic acid nanostructure vaccine of the disclosure. In some embodiments, the nucleic acid nanostructure vaccine is formulated as a pharmaceutical composition. In other embodiments, provided herein are methods of administering a nucleic acid nanostructure vaccine in an effective amount to produce a T cell immune response in the subject.

As used herein, the terms “treatment”, “treating”, and “therapy” refer to therapeutic treatment and prophylactic or preventative manipulations. The terms further include ameliorating existing symptoms, preventing additional symptoms, ameliorating or preventing the underlying causes of symptoms, preventing or reversing causes of symptoms (for example, symptoms associated with cancer or an infectious disease). Furthermore, the term “treatment” also includes the application or administration of a nucleic acid nanostructure vaccine to a subject, or an isolated tissue or cell line from a subject having a disease with the purpose to cure, heal, alleviate, relieve, alter, remedy, ameliorate, improve or affect the disease, the symptoms of disease or the predisposition toward disease.

In some embodiments, the nucleic acid nanostructure vaccine is administered to a subject prophylactically (e.g., prior to onset of a disease or prior to the subject experiencing symptoms of a disease such as cancer or an infectious disease). In some embodiments, the nucleic acid nanostructure vaccine is administered to a subject at least 1, 2, 3, 4, 5, 6, 7, 8, 9, or more weeks prior to onset of a disease. In some embodiments, the nucleic acid nanostructure vaccine is administered to a subject at least 1, 2, 3, 4, 5, 6, 7, 8, 9, or more weeks prior to the subject experiencing symptoms of a disease. In some embodiments, a prophylactic dose of a nucleic acid nanostructure vaccine provides a therapeutic benefit for at least 1, 2, 3, 4, 5, 6, 7, 8, 9, or more weeks after administration of the vaccine. In some embodiments, a prophylactic dose of a nucleic acid nanostructure vaccine provides a therapeutic benefit (e.g., prevention of onset of a disease, e.g., cancer) for at least 1, 2, 3, 4, 5, 6, 7, 8, 9, or more weeks after administration of the vaccine. In some embodiments, a prophylactic dose of a nucleic acid nanostructure vaccine provides a therapeutic benefit (e.g., prevention of onset of a disease, e.g., cancer) for 1, 2, 3, 4, 5, 6, 7, 8, 9, or more months after administration of the vaccine. In some embodiments, a prophylactic dose of a nucleic acid nanostructure vaccine provides a therapeutic benefit (e.g., prevention of onset of a disease, e.g., cancer) for 0-10, 1-10, 2-7, 2-5, 5-10, or 5-20 weeks after administration of the vaccine. In some embodiments, a prophylactic dose of a nucleic acid nanostructure vaccine provides a therapeutic benefit (e.g., prevention of onset of a disease, e.g., cancer) for at least 10, 15, 20, 30, 40, 50, 60, 70, 80, 90, 120, 150, or 200 days after administration of the vaccine.

Vaccination with a nucleic acid nanostructure vaccine of the disclosure may include administration of a single dose, or administration of two or more doses. Thus, in some embodiments, vaccination comprises administering a first dose and administering a second (booster) dose. An additional (booster) dose may be administered 1 week, 2 weeks, 3 weeks, 1 month, 2 months, 3 months, 4 months, 5 months, 6 months, 9 months, or 1 year (e.g., yearly) after a preceding (e.g., first) dose.

In some embodiments, the nucleic acid nanostructure vaccine is administered to a subject having a disease (e.g., cancer or an infectious disease). In some embodiments, the nucleic acid nanostructure vaccine is administered to a former cancer patient who has no remaining signs of disease (e.g., no observable tumor). In some embodiments, administration of the nucleic acid nanostructure vaccine to a former cancer patient provides a therapeutic benefit to the patient (e.g., no tumor remission) for at least 1, 2, 3, 4, 5, 6, 7, 8, 9, or more weeks after administration of the vaccine. In some embodiments, administration of the nucleic acid nanostructure vaccine to a former cancer patient provides a therapeutic benefit to the patient (e.g., no tumor remission) for at least 1, 2, 3, 4, 5, 6, 7, 8, 9, or more months after administration of the vaccine. In some embodiments, administration of the nucleic acid nanostructure vaccine to a former cancer patient provides a therapeutic benefit to the patient (e.g., no tumor remission) for 0-10, 1-10, 2-7, 2-5, 5-10, or 5-20 weeks after administration of the vaccine. In some embodiments, administration of the nucleic acid nanostructure vaccine to a former cancer patient provides a therapeutic benefit to the patient (e.g., no tumor remission) for at least 10, 15, 20, 30, 40, 50, 60, 70, 80, 90, 120, 150, or 200 days after administration of the vaccine.

As used herein, the term “cancer” refers to a disease state in which a population of cells within a subject has an aberrant capacity for autonomous growth or replication. In some embodiments, cancer is an abnormal state or condition (e.g., of a tissue or organ) characterized by proliferative cell growth. A cancer may include any solid or liquid, benign or malignant, non-invasive or invasive cancer or tumor, including hyperplasias, neoplasms, carcinoma, sarcoma, a hematopoietic neoplastic disorder (e.g., leukemia), and pre-cancerous or premalignant lesions. Subjects having cancer may have symptoms such as a lump, abnormal bleeding, prolonged cough, unexplained weight loss, a change in bowel movements, genetic mutations, fusion genes, and numerical chromosome changes. In some embodiments, a cancer is colorectal cancer, prostate cancer, breast cancer, lung cancer, kidney cancer, pancreatic cancer, melanoma, bladder cancer, non-Hodgkin lymphoma, thyroid cancer, or brain cancer.

As used herein, the term “infectious disease” refers to a disease state caused by foreign organisms (e.g., microorganisms) that enter a subject, multiply, and cause a response (e.g., an inflammatory response) in the subject. In some embodiments, an infectious disease is caused by a virus (or viral particle), bacteria, or fungus.

In some embodiments, provided herein are combination methods of treatment. In such combination methods, a subject is administered a nucleic acid nanostructure vaccine of the disclosure and a second therapeutic agent. In some embodiments, the second therapeutic agent is an anti-cancer drug. An anti-cancer drug may include checkpoint inhibitors (e.g., anti-PD-1, anti-PD-L1, anti-CTLA4, anti-TIM-3, anti-LAG-3); targeted kinase inhibitors (e.g., Imatinib mesylate, Ibrutinib, Neratinib, Palpociclib, Erlotinib, Lapatinib); antibodies (e.g., Bevacizumab, Trastuzumab, Rituximab, Cetuximab); chemotherapeutics such as deoxycytidine, pyrimidine, or purine analogues (e.g., irinotecan, 5-flurouracil, lenalidomide, capecitabine, docetaxel), antibody-drug conjugates (e.g., ado-trastuzumab emtansine), or any other anti-cancer drug known to a person of ordinary skill in the art. In some embodiments, the second therapeutic agent is an anti-PD-L1 antibody.

In some embodiments, the present disclosure provides methods for manipulating, directly in the body, dendritic-cell recruitment and activation. Immature dendritic cells patrol peripheral tissues, and on uptake of foreign substances (e.g., antigen), they may mature to express on their surface molecules (e.g., the receptor CCR7 and major histocompatibility complex (MHC) antigen) to facilitate lymph-node homing and subsequent antigen presentation to T-cells, respectively. Elements of infection that mobilize and activate dendritic cells include inflammatory cytokines, and “danger signals” related specifically to the infectious agent. Cytosine-guanosine oligonucleotide (CpG-ODN) sequences are uniquely expressed in bacterial DNA, and are potent danger signals that stimulate mammalian dendritic-cell activation and dendritic-cell trafficking. Thus, in some embodiments, the present disclosure provides methods for administering to a subject nucleic acid nanostructures that comprise antigen (e.g., cancer antigen) and danger signals (e.g., CpG oligonucleotides).

A “subject” to which administration is contemplated includes, but is not limited to, humans (e.g., a male or female of any age group, e.g., a pediatric subject (e.g., infant, child, adolescent) or adult subject (e.g., young adult, middle-aged adult or senior adult)) and/or other non-human animals, for example mammals (e.g., primates (e.g., cynomolgus monkeys, rhesus monkeys), including commercially relevant mammals such as cattle, pigs, horses, sheep, goats, cats, and/or dogs), birds (e.g., commercially relevant birds such as chickens, ducks, geese, and/or turkeys), reptiles, amphibians, and fish. In some embodiments, the non-human animal is a mammal. The non-human animal may be a male or female and at any stage of development. A non-human animal may be a transgenic animal.

Nucleic acid nanostructures and compositions containing nucleic acid nanostructures may be administered to a subject (e.g., a human or non-human subject) intratumorally, intramuscularly, subcutaneously, intravenously (e.g., single/multiple injection(s) or continuous infusion), or by other means.

In some embodiments, nucleic acid nanostructures are administered to a subject as a component of a polymeric gel composition. The polymeric gel composition may be biocompatible and/or biodegradable. In some embodiments, the polymeric gel composition is formed from, and/or comprises at least one polylactic acid, polyglycolic acid, PLGA polymers, alginates and alginate derivatives, gelatin, collagen, agarose, natural and synthetic polysaccharides, polyamino acids such as polypeptides particularly poly(lysine), polyesters such as polyhydroxybutyrate and poly-epsilon-caprolactone, polyanhydrides; polyphosphazines, poly(vinyl alcohols), poly(alkylene oxides) particularly poly(ethylene oxides), poly(allylamines) (PAM), poly(acrylates), modified styrene polymers such as poly(4-aminomethylstyrene), pluronic polyols, polyoxamers, poly(uronic acids), poly(vinylpyrrolidone) and copolymers of the above, including graft copolymers (see, e.g., International Publication No. WO2009102465).

The nucleic acid nanostructure vaccines are administered in effective amounts to provide a therapeutic benefit (e.g., treatment, e.g., prevention of tumor formation or reduction of tumor volume) without undue adverse effects. The dose of nucleic acid nanostructure vaccines required to achieve a particular therapeutic benefit will vary based on several factors including, but not limited to: the route of administration, the specific disease or disorder (e.g., specific type of cancer) being treated, and the stability of the vaccine (or combination therapy). One of skill in the art can readily determine a dose of nucleic acid nanostructure vaccine required and necessary to achieve a particular therapeutic benefit in a subject having a particular disease or disorder based on the aforementioned factors, as well as other factors that are well known in the art.

Delivery vehicles such as liposomes, nanocapsules, microparticles, microspheres, lipid particles, vesicles, and the like, may be used for administration of the nucleic acid nanostructure vaccines of the disclosure into suitable host cells. The formation and use of liposomes is generally known to those of skill in the art. Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

### Additional Embodiments

Additional embodiments of the present disclosure are provided in the following numbered paragraphs:
1. A nucleic acid Barrel nanostructure conjugated to one or more antigen molecules and one or more adjuvant molecules.
2. The nucleic acid Barrel nanostructure of paragraph 1, wherein the distance between any two adjacent adjuvant molecules is 2 nm to 10 nm.
3. The nucleic acid Barrel nanostructure of paragraph 2, wherein the distance between any two adjacent adjuvant molecules is 4-10 nm, optionally 4-5 nm, 4-6 nm, 4-7 nm, or 4-8 nm.
4. The nucleic acid Barrel nanostructure of paragraph 3 or 4, wherein the distance between any two adjacent adjuvant molecules is about 4.3 nm.
5. The nucleic acid Barrel nanostructure of any one of the preceding paragraphs, wherein the density of adjuvant molecules on the nucleic acid nanostructure is 1 molecule per 5 to 50 nm².
6. The nucleic acid Barrel nanostructure of paragraph 5, wherein the density of adjuvant molecules on the nucleic acid nanostructure is 1 molecule per 10 to 20 nm², or 1 molecule per 20 to 30 nm².
7. The nucleic acid Barrel nanostructure of any one of the preceding paragraphs, wherein the antigen molecules and/or the adjuvant molecules are located on a surface of the nucleic acid nanostructure.
8. The nucleic acid Barrel nanostructure of any one of the preceding paragraphs, comprising 5 to 25, 10 to 25, or 15 to 25 adjuvant molecules.
9. The nucleic acid Barrel nanostructure of any one of the preceding paragraphs, wherein the antigen molecules are covalently conjugated to the nanostructure.
10. The nucleic acid Barrel nanostructure of any one of the preceding paragraphs, comprising DNA, RNA, or a mixture of DNA and RNA.
11. The nucleic acid Barrel nanostructure of paragraph 10, wherein the nucleic acid of the nanostructure comprises or consists of DNA.
12. A method comprising administering to a subject the nucleic acid Barrel nanostructure of any one of the preceding paragraphs in an effective amount to produce a T cell immune response in the subject.
13. The method of paragraph 12, wherein the subject has a tumor.
14. The method of paragraph 13, wherein the antigen molecules are tumor antigen molecules.
15. The method of any one of the preceding paragraphs, wherein administration of the nanostructure stimulates a stronger Th1 immune response, relative to stimulation of a Th2 response.
16. The method of any one of the preceding paragraphs, wherein the administering is intratumoral, subcutaneous, intramuscular, or intravenous.

### EXAMPLES

Previously, DNA origami-based square-lattice blocks (SQBs) that control adjuvant spacing in the context of loaded antigens were successfully assembled. Through in vitro immune cell culture studies and in vivo tumor-treatment models, data demonstrated that square blocks induce Th1 immune polarization when CpG is spaced at 3.5 nm. Low doses of this DNA origami cancer vaccine enhanced antigen cross-presentation, CD8⁺ T cell activation and Th1-polarized CD4⁺ T cell activation. The vaccine also synergized with anti-PD-L1 for effective cancer regression in both melanoma and lymphoma. See International Publication Number WO 2020/247724, incorporated herein in its entirety.

### Example 1: Basic Barrel Design and Modification and Folding Optimization

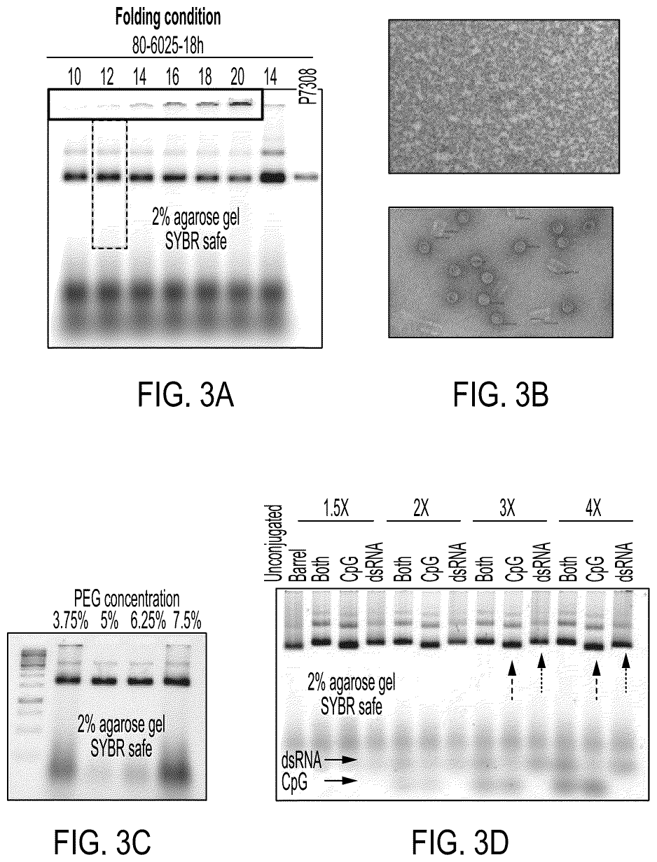

A barrel structure design, as shown in FIGS. 2A-2B was used to produce a vaccine platform. The barrel was folded using bacteriophage M13 single-stranded DNA scaffold (7308 nt) complemented to hundreds of staple strands. Some mini scaffold was also applied for the design. All the design was done by honeycomb Cadnano software. There are three layers of double helices (FIG. 1), outer (14 double helices), middle and inner layer (15 double helices). The barrel was self-assembled under a buffer condition of 1×TE, 12 mM MgCl₂. Based on the basic barrel structure, we designed different modification sites for CpG, dsRNA, dye and coiled-coil peptide (FIGS. 2A-2B). We have also determined the folding condition, purification condition and payload modification condition for the barrel (FIGS. 3A-3C). In some embodiments, the temperature ramp for Barrel was denaturing at 80 degrees for 15 min, and then decreasing from 65 to 25 degrees over 18 hours. An example barrel purification condition was 5% PEG solution in the final mixture, mixing the barrel and 10% PEG solution at a 1:1 volume ratio and keep the MgCl₂ at 12 mM. For CpG and dsRNA conjugation, the adjuvants were added at a two-fold excess, which provided complete conjugation efficiency. The barrel production is highly efficient, repeatable, and scalable.

### Example 2: Barrel Vaccine Platform Fabrication

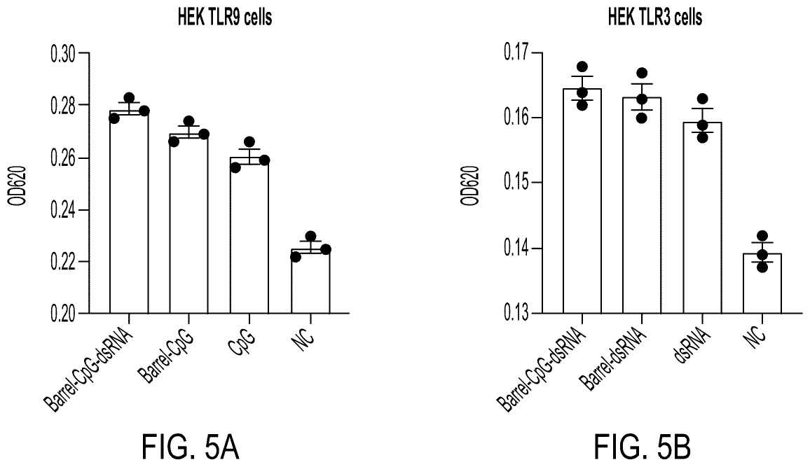

We successfully fabricated adjuvant CpG and dsRNA (a TLR3 ligand) on the Barrel with a spacing 4.3 nm between adjacent adjuvant molecules (FIGS. 4A-4D). By coculturing the Barrel origami fabricated with CpG and dsRNA with TLR9 (CpG receptor) or TLR3 (dsRNA receptor) expressing HEK blue cells, we detected improved receptor activation compared to the free adjuvant (FIGS. 5A-5B). 1 nM Barrel conjugated to (1) both CpG and dsRNA (“Barrel-CpG-dsRNA”), (2) CpG only (“Barrel-CpG”), or (3) dsRNA only (“Barrel-dsRNA”) were used for the cell stimulation over 24 hours. The corresponding concentrations of CpG and dsRNA were 24 nM. In the free adjuvant controls (i.e., the adjuvant(s) are not conjugated to a Barrel), 24 nM CpG (“CpG”) or 24 nM dsRNA (“dsRNA”) were applied. A negative control (no adjuvants or Barrel, “NC”) was also tested.

Additionally, we identified several candidates coiled peptides (CCPs), namely E4_N, K4_N, K/E4_N, E4_C and E3_N, as potential neoantigen capturing motifs (FIG. 6A). N or C refers to the terminus connecting to the 42 DNA handles as shown in FIG. 2C. These peptides form stable alpha helices with a hydrophilic and hydrophobic faces. The hydrophobic face readily binds/interacts with hydrophobic faces of peptides or proteins. We have successfully conjugated the neoantigen-capturing CCPs inside the Barrel to complete the DNA origami antigen-capturing platform (FIGS. 6B-6E). E4_N, K/E4_N, and E4_C behaved well during the fabrication process (FIG. 6D). TEM results verified the successful conjugation of all the components (FIG. 6E). Gel analysis verified that the coiled-coil peptides could capture excess coiled-coil peptides once they were attached inside the Barrel, increasing with more excess (from 0.5 to 4 times) of CCPs conjugated barrel (FIGS. 6F, 6G). These results provide validation for the Barrel vaccine platform.

### Example 3: Barrel Vaccine Nanoparticles Captured Short Peptides

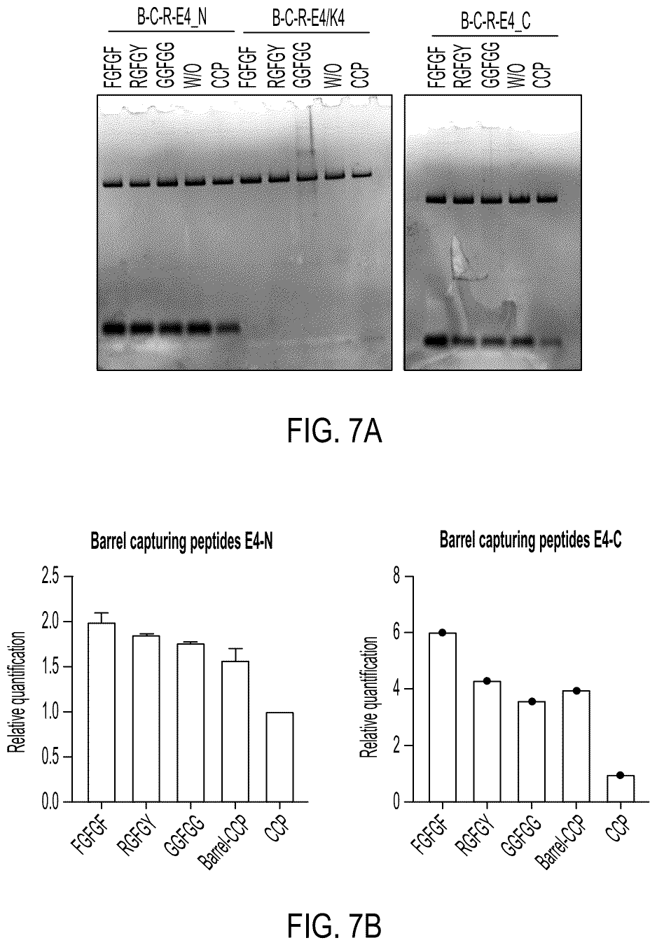

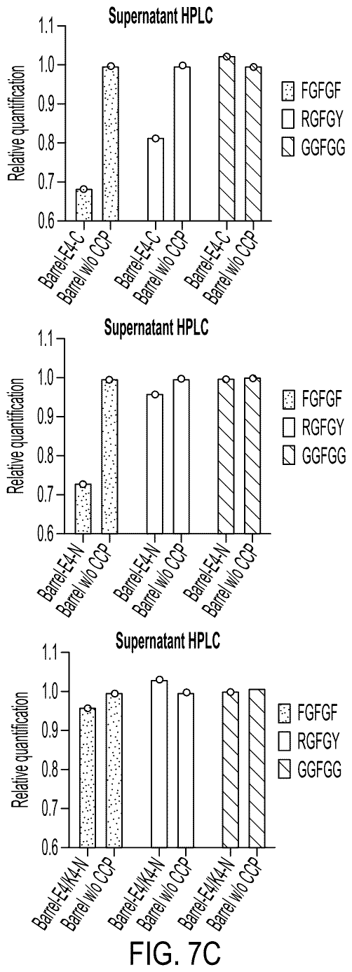

Next, we tested the ability of different coiled-coil peptides (CCPs) to capture short peptides with different hydrophobicities. Barrel origami was conjugated to the E4_N, E4_C, or E4/K4_N CCP and exposed to short peptide sequences (FGFGF, RGFGY, or GGFGG). Overall, the hydrophobicity level is FGFGF>RGFGY>GGFGG. Our results showed that both CCP E4_N and CCP E4_C could capture more of the hydrophobic FGFGF peptide compared to other peptides (FIGS. 7A-7B). E4/K4_N captured few peptides, possibly due to its neutral charge. We also tested the supernatant after the Barrel-CCP captured the short peptides and was precipitated from solution with PEG. HPLC results showed that the concentration of peptides remaining in the supernatant increased while the hydrophobicity of the peptides decreased (FIG. 7C). These data demonstrate that coiled-coil peptides are capable of preferentially capturing hydrophobic peptides.

### Example 4: Barrel Vaccine Platform Successfully Captured Protein Released from the Irradiated Tumor Cells

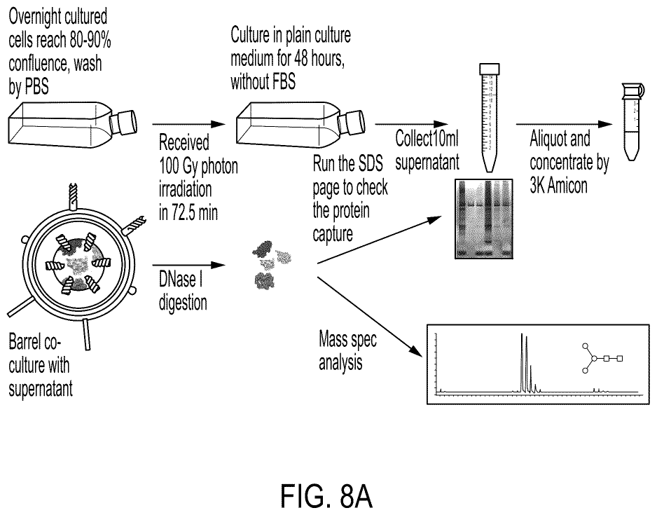

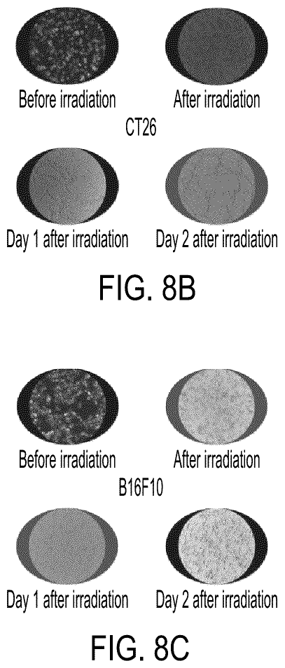

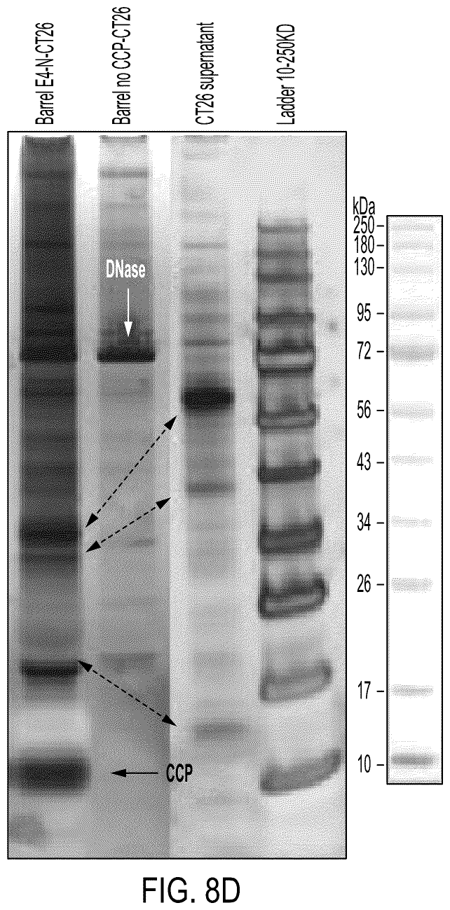

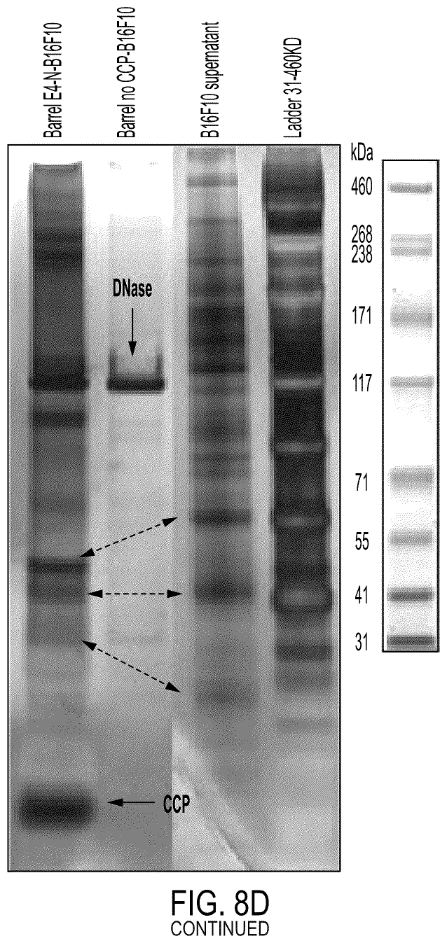

To demonstrate if the Barrel vaccines could capture the proteins released from tumor cells following immunogenic cell death (ICD), we irradiated CT26 colon carcinoma and B16F10 melanoma tumor cell line at 100 Gy photons and concentrated the culture supernatant by MWCO filtration (FIGS. 8A-8C). We cocultured the Barrel vaccines with the supernatant. After that, we did PEG purification of the Barrel and then applied DNase I digestion. The DNase I digestion will remove the barrel structure but keep the protein that's been captured for detection. The protocol for this Example is as described in FIG. 8A. We ran the sample in SDS page gel and did the silver stain to verify that Barrel-E4_N could successfully capture the proteins secreted by irradiated cells (FIG. 8D). These results demonstrate that CCPs should capture antigens from tumor ICD.

### Example 5: Mass Spectrometry Analysis of the Captured Proteins

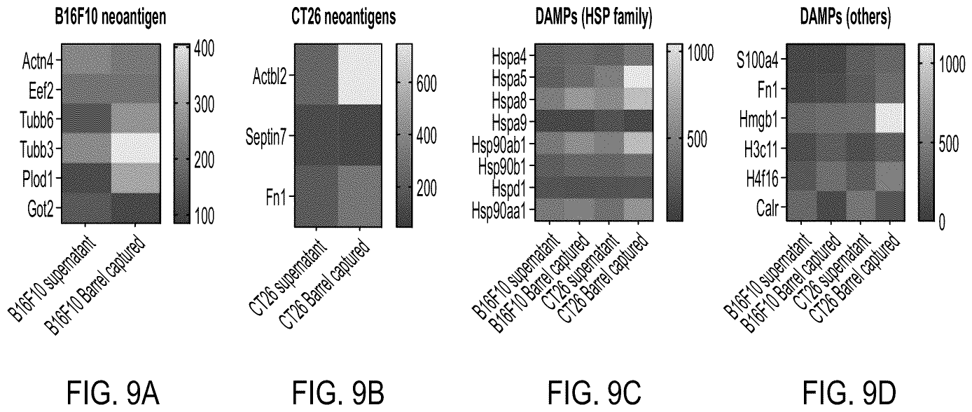

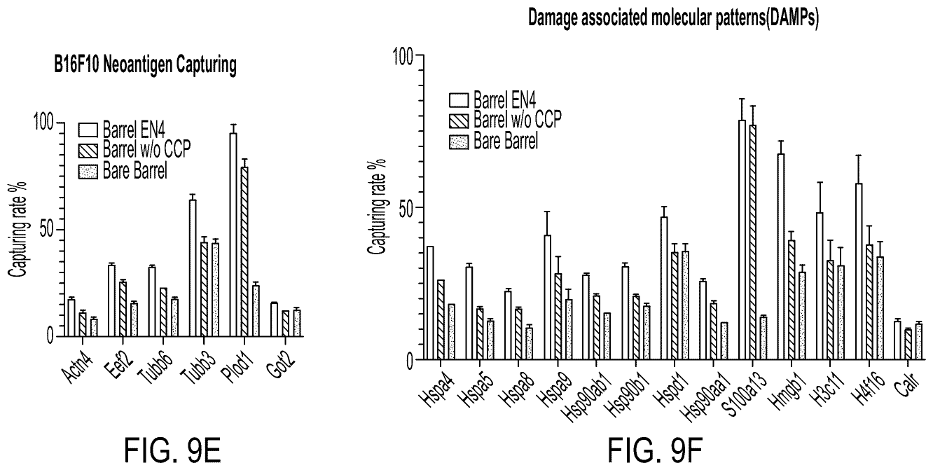

We have verified that the Barrels (conjugated to a coiled-coil peptide) could capture the proteins secreted by irradiated cells. Next, we used mass spectrometry to understand what antigens have been captured. For both B16F10 and CD26, we showed that several tumor neoantigens were captured by the Barrels (FIGS. 9A-9B). Specifically, it was found that the Barrels (conjugated to a coiled-coil peptide) captured Actn4, Eef2, Tubb6, Tubb3, Plod1, and Got2 in the B16F10 model (FIG. 9A); and that the Barrels (conjugated to a coiled-coil peptide) captured Actb12, Septin7, and Fn1 in the CT26 model. We also identified heat shock proteins and other DAMP-related proteins highly enriched among the Barrel-captured proteins (FIGS. 9C-9D) such as Hspa5, Hspa8, Hsp90b1, Hsp90aa1, Hmgb1, and H4f16. In a more sophisticated exploration for B16F10, we compared the protein abundance in different barrel conditions, and verified that only when CCP is included in the Barrel, the antigen capturing efficiency is the highest (FIG. 9E-9F).

### Example 6: Barrel DNA Origami Vaccine (DoriVac) Showed Efficacy on Highly Resistant Tumor Models

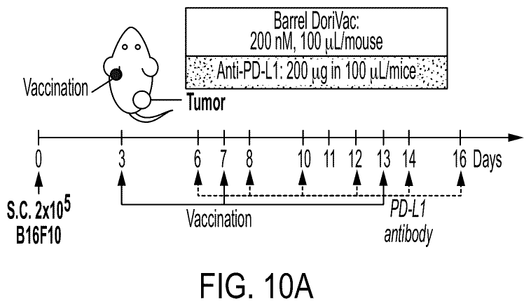

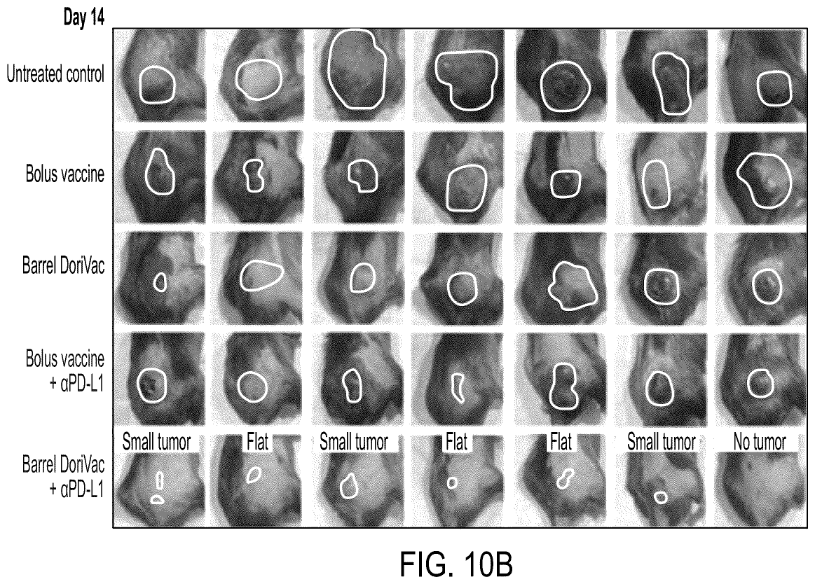

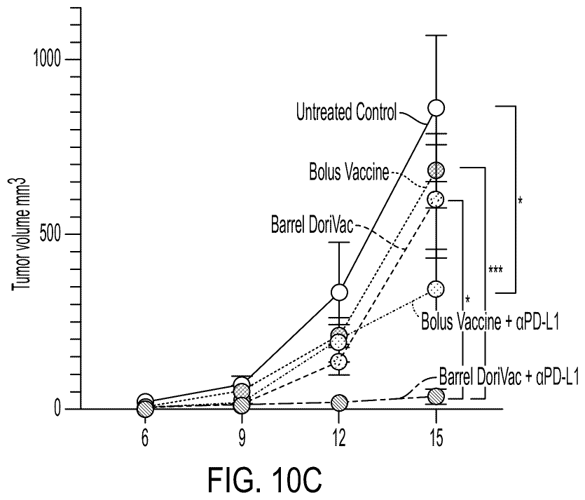

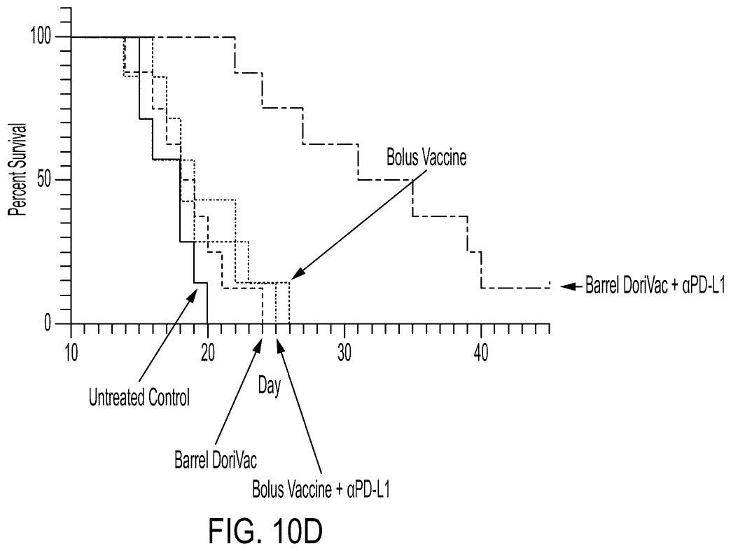

Results in this example showed that Barrel DoriVac with captured antigens in combination with a therapeutically effective anti-PD-L1 antibody promotes robust and durable tumor control in the highly resistant B16F10 melanoma model and another colon carcinoma model (FIG. 10-FIG. 14).

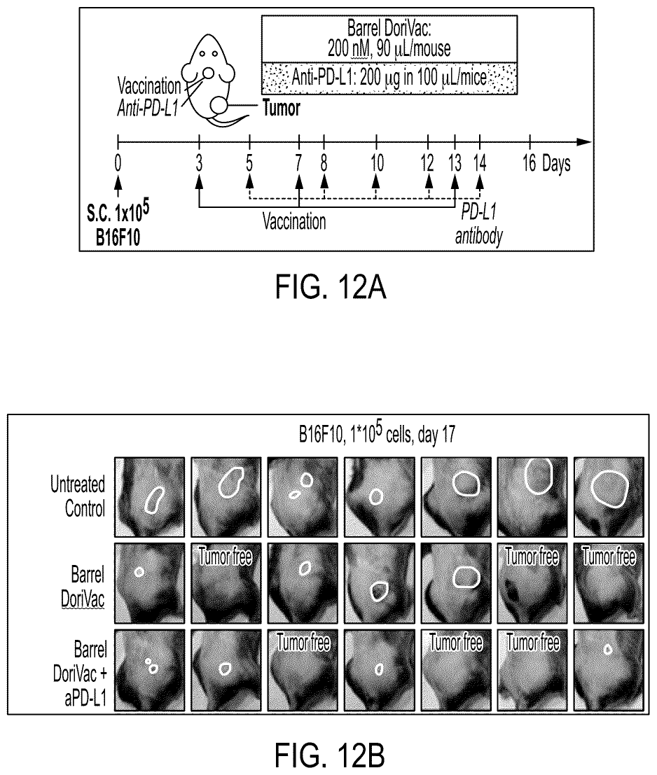

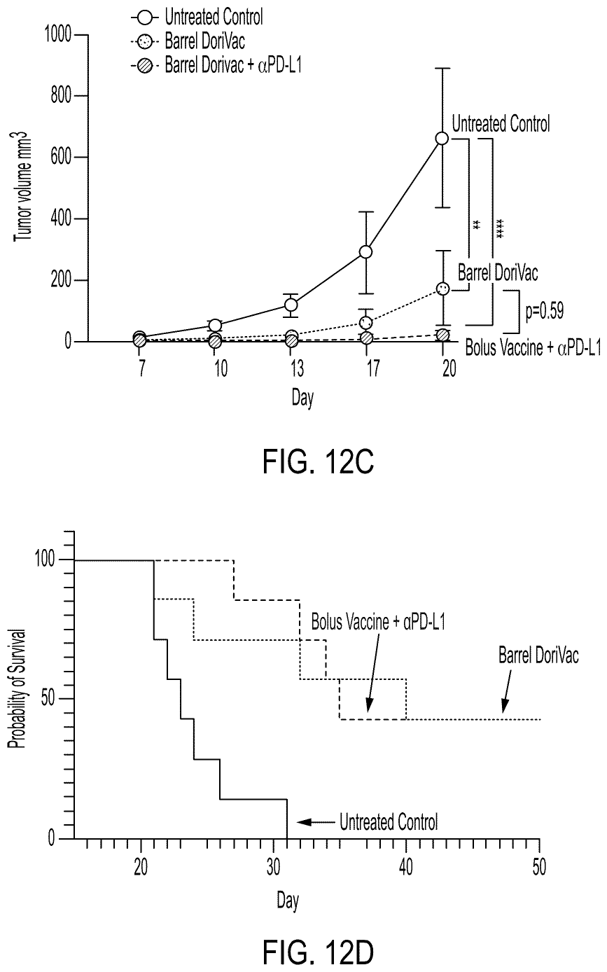

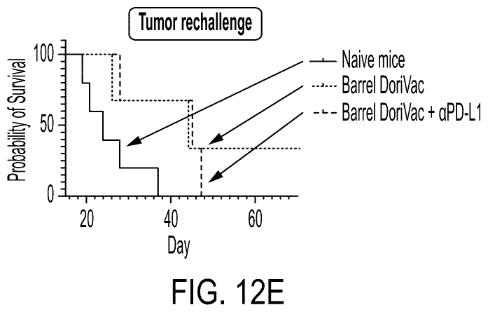

To use the Barrel DoriVac as a whole vaccine, the vaccine fabrication was done before applying to a subject (a mouse subject). First, a Barrel was conjugated to adjuvant and antigen-capturing motifs (e.g., coiled-coil peptides). The Barrel was then exposed to antigens to allow for antigen capturing by Barrel, which was validated by silver stain as described previously. The B16F10 tumor model was set up by injecting 200K or 100K tumor cells to the right flank of the mice. The vaccine treatment (Barrel DoriVac or bolus (tumor supernatant, free CCP and free CpG and dsRNA adjuvant (no Barrel nanostructure)) was applied subcutaneously on the left shoulder on day 3, 7 and 13 after tumor inoculation, and the anti-PD-L1 was given on day 6, 8, 10, 12, 14 and 16 subcutaneously at the same location of vaccine administration (FIGS. 10A, FIG. 12A). Mouse tumors and survival was recorded.

It was found that the combination treatment of Barrel DoriVac and anti-PD-L1 antibody could significantly inhibit the tumor growth and prolong mouse survival (FIGS. 10B-10C, FIGS. 12B-12C).

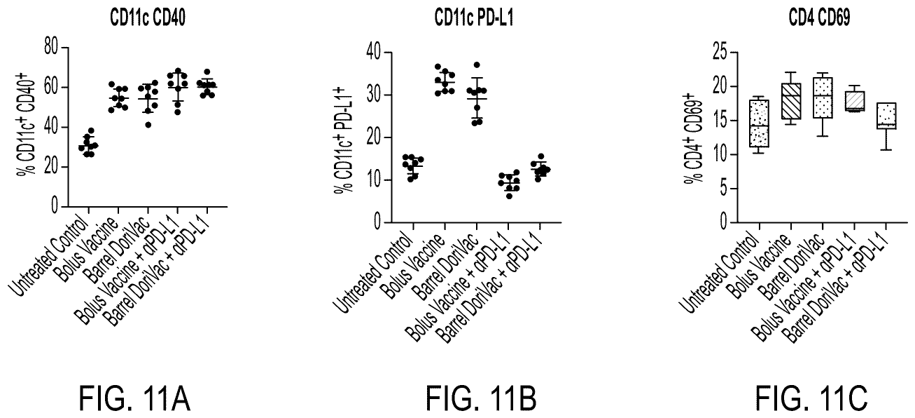

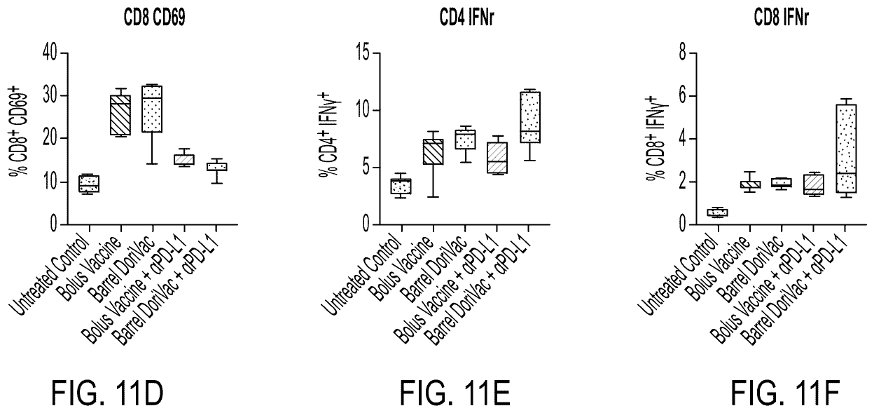

In mice dosed with 200K tumor cells, there was a significant reduction in tumor volume (FIG. 10C) and a prolonged lifespan (select mice treated with the Barrel DoriVac and the anti-PD-L1 antibody were able to survive out to at least 40 days, while mice in the other treatment groups were all dead before Day 30) (FIG. 10D) for mice treated with the combination of the Barrel DoriVac and the anti-PD-L1 antibody. For the 200K B16F10 model, we also determined the immune cell profile of the mice. This data demonstrated that mice treated with the combination of the Barrel DoriVac and the anti-PD-L1 antibody had lowered levels of PD-L1⁺ cells relative to mice treated with the Barrel DoriVac alone (PD-L1 inhibition) (FIG. 11B). Mice treated with the combination treatment also experienced enhanced CD4 and CD8 T cell activation by IFNγ expression (FIGS. 11E-11F).

When we used a less aggressive B16F10 tumor model (100K cells) (FIG. 12A), Barrel DoriVac alone and the combination of Barrel DoriVac and anti-PD-L1 antibody also significantly reduced tumor volume (FIG. 12C) and provided a prolonged lifespan (with 3 of 7 mice in each treatment group surviving to the end of the experiment at 50 days) (FIG. 12D).

Mice that survived for 50 days after treatment with DoriVac or DoriVac/anti-PD-L1 antibody (n=3 for each treatment group) in the B16F10 tumor model (100K cells) were re-challenged with a fresh inoculation of 1×10⁵ B16F10 tumor cells. Naïve mice receiving the same amount of cells were used as a control group. As shown in FIG. 12E, all of the mice in the two treatment groups survived for approximately 25 days following the tumor re-challenge, with two mice in each treatment group surviving beyond 40 days.

These data demonstrate that a nucleic acid Barrel nanostructure vaccine as described herein (e.g., conjugated to adjuvant molecules and antigen-capturing motifs) is capable of reducing tumor volumes and treating cancer (in tested models) by prolonging lifespan. These beneficial effects can be shown when using the nucleic acid Barrel nanostructure vaccine alone or in combination with another cancer treatment molecule (e.g., anti-PD-L1 antibody). Furthermore, the tumor re-challenge data demonstrates that the nucleic acid Barrel nanostructure vaccine is capable of providing functional benefit to subjects to treat initial tumors and to prevent tumor recurrence.

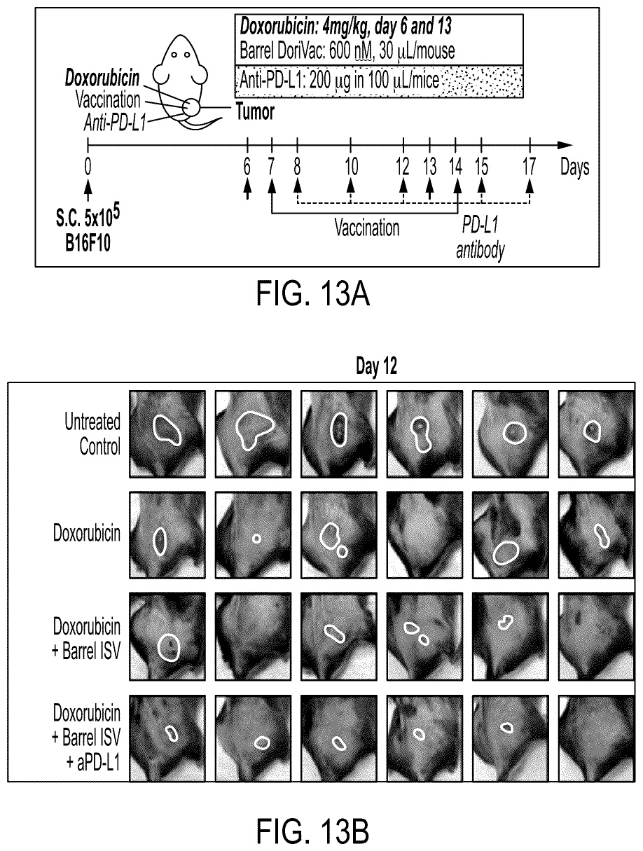

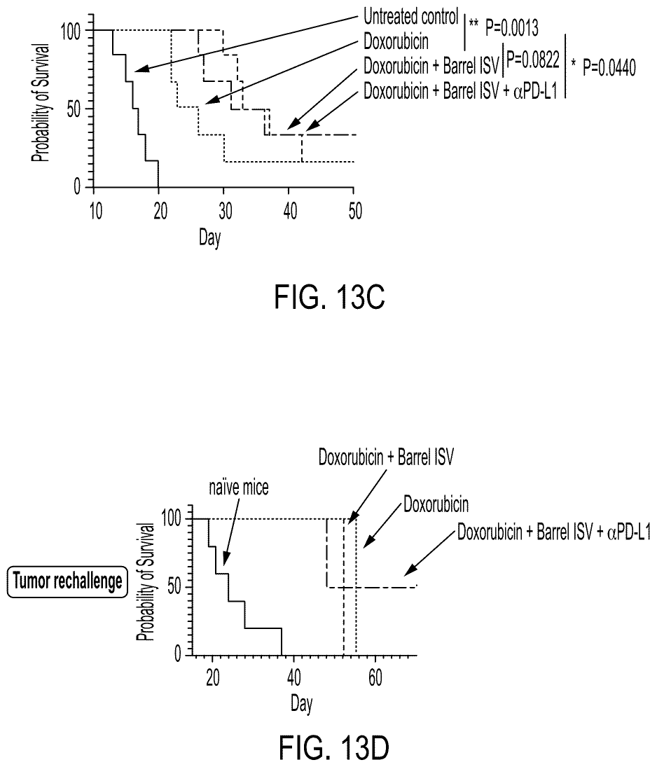

We also tried to understand if Barrel DoriVac can capture the antigen locally after immunogenic cell death (ICD) was induced by a chemotherapeutic drug (FIG. 13). Doxorubicin was applied 6 days after tumor inoculation (500K B16F10 cells) when the tumor reached 50-80 mm2, Barrel vaccination platform was applied following the ICD intratumorally. Anti-PD-L1 was applied to the site surrounding the tumor tissue (FIG. 13A). We could see the synergy of the Doxorubicin ICD and Barrel vaccination platform (FIGS. 13B-13C). Mice exposed to doxorubicin and subsequently treated with the Barrel DoriVac (“Barrel ISV”) or the combination of Barrel DoriVac and anti-PD-L1 antibody (“Barrel ISV+αPD-L1”) had prolonged rates of survival relative to untreated mice exposed to doxorubicin (FIG. 13C). Several mice survived to the end of the experiment (at 50 days). Mice that survived for 50 days after treatment with DoriVac or DoriVac/anti-PD-L1 antibody were re-challenged with a fresh inoculation of 1×10⁵ B16F10 tumor cells. Naïve mice receiving the same amount of cells were used as a control group. As shown in FIG. 13D, mice treated with DoriVac demonstrated prolonged rates of survival, with several mice in the DoriVac treatment surviving for approximately 70 days following the tumor re-challenge.

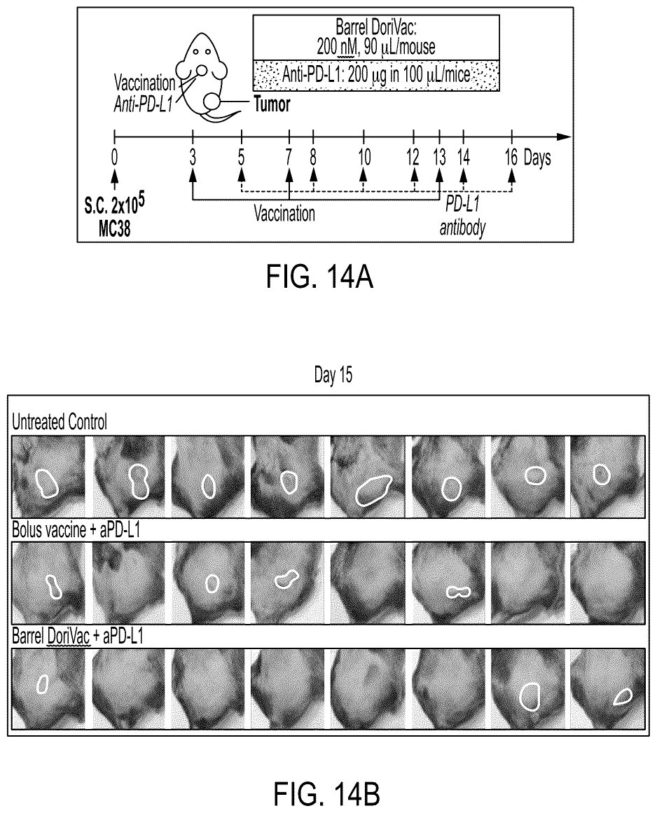

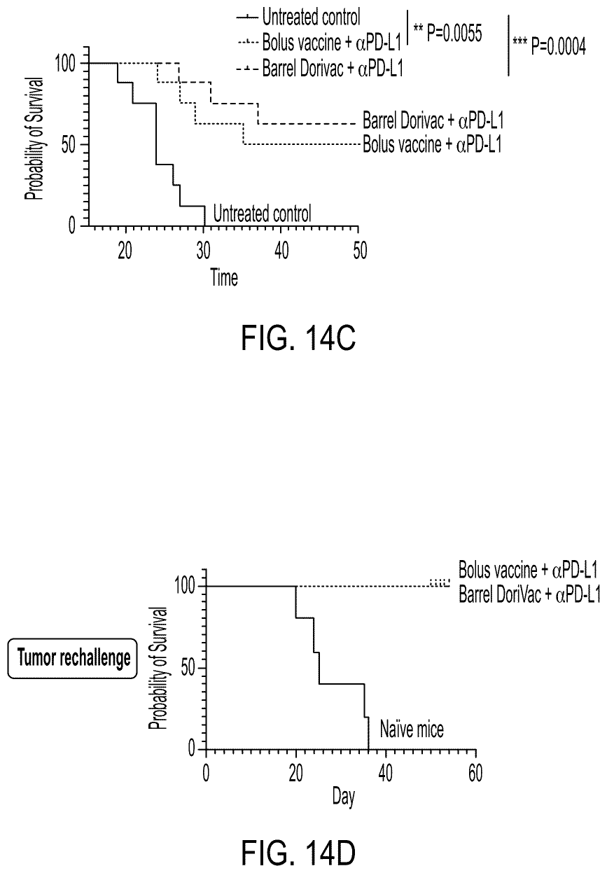

The efficacy of Barrel DoriVac in MC38 colon carcinoma model was also tested. The MC38 tumor model was set up by injecting 200K tumor cells to the right flank of the mice. The vaccine treatment (Barrel DoriVac or bolus (no Barrel)) was applied subcutaneously on the left shoulder on day 3, 7 and 13 after tumor inoculation, and the anti-PD-L1 was given on day 6, 8, 10, 12, 14 and 16 subcutaneously at the same location of vaccine administration (FIG. 14A). Mouse tumors and survival was recorded. The Barrel DoriVac with anti-PD-L1 treatment cured 5 out of 8 mice and showed a prolonged mouse survival (FIG. 14C). Mice that survived for 50 days after treatment with DoriVac/anti-PD-L1 antibody or Bolus vaccine/anti-PD-L1 antibody were re-challenged with a fresh inoculation of 2×10⁵ MC38 tumor cells. Naïve mice receiving the same amount of cells were used as a control group. As shown in FIG. 14D, none of the mice treated with DoriVac died as a result of the re-challenge, demonstrating the persistent function of this vaccine.

### Example 7: Barrel DNA Origami Vaccine (DoriVac) Showed Efficacy as a Prophylactic Vaccine

Barrel DoriVac (nucleic acid Barrel nanostructure with antigen-capturing coiled-coil peptides, and adjuvants) was prepared as previously described in Example 6. C57BL6 mice received two doses of DoriVac, Bolus vaccine (tumor supernatant, free CCP, free CpG, and dsRNA adjuvant), or saline (control) on Day 0 and Day 7. 1×10⁵ B16F10 cells or 2×10⁵ MC38 cells were applied to inoculate the right flank of the mice on Day 7. Mouse survival was recorded (n=5).

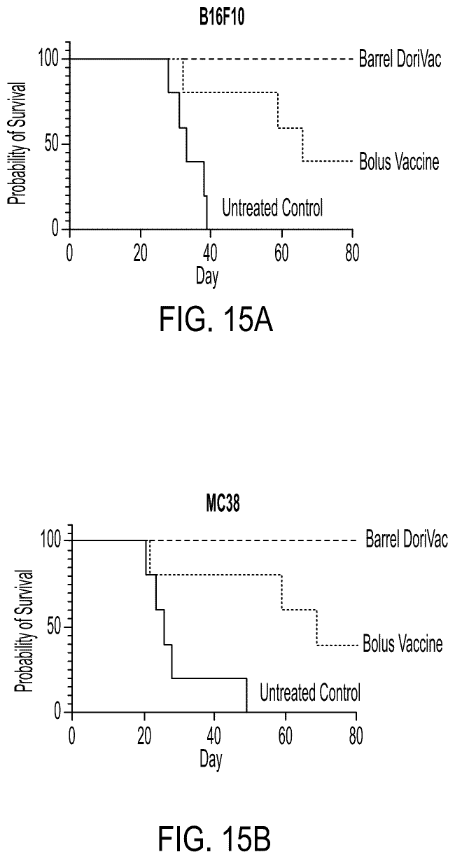

As shown in FIG. 15A, all of the mice that were prophylactically treated with DoriVac vaccine prior to challenge with B16F10 tumor cells survived to the end of the experimental period (Day 80). Fewer than 50% of mice treated with Bolus vaccine survived to the end of the experimental period; and none of untreated control mice survived past Day 40.

As shown in FIG. 15B, all of the mice that were prophylactically treated with DoriVac vaccine prior to challenge with MC38 tumor cells survived to the end of the experimental period (Day 80). Fewer than 50% of mice treated with Bolus vaccine survived to the end of the experimental period; and none of untreated control mice survived past Day 50.

These data demonstrate that prophylactic administration (i.e., administration that occurs prior to cancer initiation) of a nucleic acid Barrel nanostructure vaccine as described herein (e.g., conjugated to adjuvant molecules and antigen-capturing motifs) can be useful in treating cancer (by prolonging lifespan). Specifically, prophylactic administration of nucleic acid Barrel nanostructure vaccines as described herein are capable of prolonging the average lifespan of subjects having cancer relative to a cancer vaccine that does not comprise a nucleic acid Barrel nanostructure.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

What is claimed is:

1. A nucleic acid Barrel nanostructure conjugated to adjuvant molecules and antigen-capturing motifs.

2. A nucleic acid Barrel nanostructure conjugated to antigen molecules and adjuvant molecules.

3. The nucleic acid Barrel nanostructure of claim 2 further comprising antigen-capturing motifs.

4. The nucleic acid Barrel nanostructure of any one of the preceding claims, wherein the distance between any two adjacent adjuvant molecules is 2 nm to 10 nm.

5. The nucleic acid Barrel nanostructure of claim 4, wherein the distance between any two adjacent adjuvant molecules is 4-10 nm, optionally 4-5 nm, 4-6 nm, 4-7 nm, or 4-8 nm.

6. The nucleic acid Barrel nanostructure of claim 4 or 5, wherein the distance between any two adjacent adjuvant molecules is about 4.3 nm.

7. The nucleic acid Barrel nanostructure of any one of the preceding claims, wherein the density of adjuvant molecules on the nucleic acid nanostructure is 1 molecule per 5 nm² to 1 molecule per 50 nm².

8. The nucleic acid Barrel nanostructure of claim 7, wherein the density of adjuvant molecules on the nucleic acid nanostructure is 1 molecule per 10 to 20 nm², or 1 molecule per 20 to 30 nm².

9. The nucleic acid Barrel nanostructure of any one of the preceding claims, wherein the antigen molecules and/or the antigen-capturing motifs and/or the adjuvant molecules are located on a surface of the nucleic acid nanostructure.

10. The nucleic acid Barrel nanostructure of any one of the preceding claims, wherein the nanostructure comprises adjuvant molecules conjugated to an exterior surface of the nanostructure.

11. The nucleic acid Barrel nanostructure of any one of the preceding claims, wherein the nanostructure comprises antigen-capturing motifs conjugated to an interior surface of the nanostructure.

12. The nucleic acid Barrel nanostructure of any one of the preceding claims, wherein the nanostructure comprises adjuvant molecules conjugated to an exterior surface of the nanostructure and antigen-capturing motifs conjugated to an interior surface of the nanostructure.

13. The nucleic acid Barrel nanostructure of any one of the preceding claims, comprising 5 to 200, 100 to 200, 100 to 150, 5 to 25, 10 to 25, or 15 to 25 adjuvant molecules.

14. The nucleic acid Barrel nanostructure of any one of claims 2-13, wherein the antigen molecules are covalently conjugated to the nanostructure.

15. The nucleic acid Barrel nanostructure of any one of the preceding claims, wherein the antigen-capturing motifs are coiled-coil peptides.

16. The nucleic acid Barrel nanostructure of claim 15, wherein the coiled-coil peptides comprise the amino acid sequence of any one of SEQ ID NOs: 1-10.

17. The nucleic acid Barrel nanostructure of any one of the preceding claims, comprising DNA, RNA, or a mixture of DNA and RNA.

18. The nucleic acid Barrel nanostructure of claim 17, wherein the nucleic acid of the nanostructure comprises or consists of DNA.

19. A composition comprising the nucleic acid Barrel nanostructure of any one of the preceding claims and a second therapeutic agent, optionally wherein the second therapeutic agent is an anti-cancer drug, further optionally wherein the anti-cancer drug is an anti-PD-L1 antibody.

20. A pharmaceutical composition for use in vaccination of a subject against a disease, wherein the pharmaceutical composition comprises the nucleic acid Barrel nanostructure of any one of claims 1-18.

21. A method comprising administering to a subject the nucleic acid Barrel nanostructure of any one of claims 1-18, the composition of claim 19, or the pharmaceutical composition of claim 20 in an effective amount to produce a T cell immune response in the subject.

22. A method of treating a subject having a disease, the method comprising administering to the subject the nucleic acid Barrel nanostructure of any one of claims 1-18, the composition of claim 19, or the pharmaceutical composition of claim 20 in an effective amount to treat the disease.

23. The method of claim 21 or 22, wherein the subject has a tumor, optionally a cancerous tumor.

24. The method of claim 23, wherein the antigen molecules are tumor antigen molecules.

25. The method of any one of claims 21-24, wherein administration of the nanostructure stimulates a stronger Th1 immune response, relative to stimulation of a Th2 response.

26. The method of any one of claims 22-25, wherein the disease is a cancer.

27. A method comprising administering to a subject the nucleic acid Barrel nanostructure of any one of claims 1-18, the composition of claim 19, or the pharmaceutical composition of claim 20.

28. The method of claim 27, wherein the nucleic acid Barrel nanostructure, composition, or pharmaceutical composition is administered in an effective amount to prevent onset of a disease, optionally to prevent onset of a disease for 1, 2, 3, 4, 5, 6, 7, 8, 9, or more months after the administration.

29. The method of any one of claims 19-28, wherein the administering is intratumoral, subcutaneous, or intravenous.


SEQUENCE LISTING (10 sequences)

SEQ ID NO: 1 - protein, 29 aa, synthetic construct
  MOD_RES 1: note = 6-Azido-L-lysine
KKIAALKEKIAALKEKNAALKWKIAALKE

SEQ ID NO: 2 - protein, 29 aa, synthetic construct
KKIAALKEKIAALKEKNAALKWKIAALKE

SEQ ID NO: 3 - protein, 29 aa, synthetic construct
  MOD_RES 1: note = 6-Azido-L-lysine
KEIAALEKEIAALEKENAALEWEIAALEK

SEQ ID NO: 4 - protein, 29 aa, synthetic construct
KEIAALEKEIAALEKENAALEWEIAALEK

SEQ ID NO: 5 - protein, 29 aa, synthetic construct
  MOD_RES 1: note = 6-Azido-L-lysine
KEIAALEQEIAALEQKNAALKWKIAALKQ

SEQ ID NO: 6 - protein, 29 aa, synthetic construct
KEIAALEQEIAALEQKNAALKWKIAALKQ

SEQ ID NO: 7 - protein, 29 aa, synthetic construct
  MOD_RES 29: note = 6-Azido-L-lysine
EIAALEKEIAALEKENAALEWEIAALEKK

SEQ ID NO: 8 - protein, 29 aa, synthetic construct
EIAALEKEIAALEKENAALEWEIAALEKK

SEQ ID NO: 9 - protein, 22 aa, synthetic construct
  MOD_RES 1: note = 6-Azido-L-lysine
KEIAALEKENAALEWEIAALEK

SEQ ID NO: 10 - protein, 22 aa, synthetic construct
KEIAALEKENAALEWEIAALEK